(19) United States
(12) Patent Application Publication
(10) Pub. No.: US 2009/0258862 A1
(43) Pub. Date: Oct. 15, 2009

(54) Niacin receptor agonists, compositions containing such compounds and methods of treatment

(76) Inventors: Steven L. Colletti, Princeton Junction, NJ (US); Hong Shen, West Windsor, NJ (US); James R. Tata, Westfield, NJ (US); Michael J. Szymonifka, Clark, NJ (US)

Correspondence Address: MERCK AND CO., INC, P O BOX 2000, RAHWAY, NJ, 07065-0907, US

(21) Appl. No.: 11/991,188

(22) PCT Filed: Aug. 25, 2006
(86) PCT No.: PCT/US06/33304

§ 371 Date: Feb. 28, 2008

Related U.S. Application Data
(60) Provisional application No. 60/712,275, filed on Aug. 29, 2005.

(51) Int. Cl. A61K 31/55; C07D 403/04; C07D 487/08; C07D 401/04; A61K 31/495

(52) U.S. Cl. 514/217.05; 544/353; 544/344; 540/599; 544/363; 514/249

(57) ABSTRACT

Compounds of the formula (I): as well as pharmaceutically acceptable salts and solvates are disclosed. The compounds are useful for treating dyslipidemias, and in particular, reducing serum LDL, VLDL and triglycerides, and raising HDL levels. Pharmaceutical compositions and methods of treatment are also included.

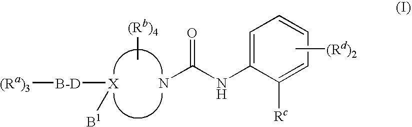

### BACKGROUND OF THE INVENTION

The present invention relates to urea compounds, compositions and methods of treatment or prevention in a mammal relating to dyslipidemias. Dyslipidemia is a condition wherein serum lipids are abnormal. Elevated cholesterol and low levels of high density lipoprotein (HDL) are associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for essential organic molecules such as steroids, cell membranes, and bile acids, cholesterol in excess is known to contribute to cardiovascular disease. For example, cholesterol is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

A compound in accordance with formula I:

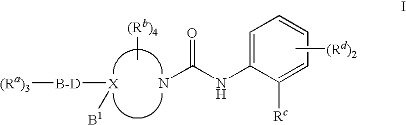

or a pharmaceutically acceptable salt or solvate thereof, is disclosed wherein:

X represents a carbon or nitrogen atom, such that

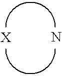

represents a 5 to 7 membered heterocyclic ring containing 1-2 nitrogen atoms;

when X represents a nitrogen atom, D represents a bond and B¹ is absent;

when X represents a carbon atom, B and B¹ can be taken together or separately;

when B and B¹ are taken together, D represents a bond and B and B¹ taken together represent a spiro ring containing 5-6 atoms, optionally containing 1 heteroatom or group selected from oxygen, sulfur, sulfinyl, sulfonyl and nitrogen, said spiro ring being optionally substituted with 1 oxo group, and optionally fused to a phenyl ring, said spiro or fused phenyl ring having 3 R^{a} groups,

and when B and B¹ are taken separately, D represents a bond, an oxygen atom or —(CH₂)₁₋₃—, B¹ represents hydrogen and

B represents a 6-10 membered aryl or a 5-10 membered heteroaryl group containing from 1-4 heteroatoms, 0-4 of which are nitrogen, 0-2 of which are oxygen and 0-1 of which are sulfur;

3 R^{a} groups are present, 1-3 of which are selected from the group consisting of: hydrogen and halo, and 0-2 of which are selected from the group consisting of:

OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;

phenyl, heteroaryl, —O-phenyl and —O-heteroaryl, said phenyl and heteroaryl groups and portions being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy OH; NH₂ and CN;

and C₁₋₃alkyl and OC₁₋₃alkyl, the alkyl portions of which are optionally substituted with 1-3 halo atoms and 1 phenyl or heteroaryl group, said phenyl and heteroaryl being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH, NH₂ and CN;

each R^{b} independently represents hydrogen, halo, C₁₋₃alkyl, haloC₁₋₃alkyl, OC₁₋₃alkyl, haloC₁₋₃alkoxy or OH, or two R^{b} groups may be combined to form a fused 5-6 membered ring, with two such rings being possible;

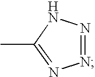

R^{c} represents —CO₂H or

and each R^{d} independently represents H, halo, methyl, or methyl substituted with 1-3 halo atoms.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means a mono- or bicyclic aromatic ring or ring system containing at least one heteroatom selected from O, S and N, with each ring containing 5 to 6 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzoisothiazolyl, benzimidazolyl, benzofuranyl, benzothiophenyl, benzopyrazolyl, benzotriazolyl, furo(2,3-b)pyridyl, quinolyl, indolyl, isoquinolyl, isoindolyl, quinoxalinyl, quinazolinyl, naphthyridinyl, pteridinyl and the like. Heteroaryl also includes aromatic carbocyclic or heterocyclic groups fused to heterocycles that are non-aromatic or partially aromatic such as indolinyl, dihydrobenzofuranyl, dihydrobenzothiophenyl, dihydrobenzoxazolyl, and aromatic heterocyclic groups fused to cycloalkyl rings. Heteroaryl also includes such groups in charged form, e.g., pyridinium.

“Heterocyclyl” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, 2,3-dihydrofuro(2,3-b)pyridyl, tetrahydrofuranyl, benzoxazinyl, 1,4-dioxanyl, tetrahydrohydroquinolinyl, tetrahydroisoquinolinyl, dihydroindolyl, morpholinyl, thiomorpholinyl, tetrahydrothienyl and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). Heterocyclyl moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

One aspect of the invention relates to compounds in accordance with formula I:

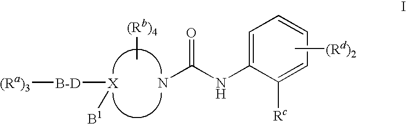

or a pharmaceutically acceptable salt or solvate thereof, is disclosed wherein:

X represents a carbon or nitrogen atom, such that

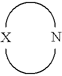

represents a 5 to 7 membered heterocyclic ring containing 1-2 nitrogen atoms;

when X represents a nitrogen atom, D represents a bond and B¹ is absent;

when X represents a carbon atom, B and B¹ can be taken together or separately;

when B and B¹ are taken together, D represents a bond and B and B¹ taken together represent a spiro ring containing 5-6 atoms, optionally containing 1 heteroatom or group selected from oxygen, sulfur, sulfinyl, sulfonyl and nitrogen, said spiro ring being optionally substituted with 1 oxo group, and optionally fused to a phenyl ring, said spiro or fused phenyl ring having 3 R^{a} groups,

and when B and B¹ are taken separately, D represents a bond, an oxygen atom or —(CH₂)₁₋₃—, B¹ represents hydrogen and

B represents a 6-10 membered aryl or a 5-10 membered heteroaryl group containing from 1-4 heteroatoms, 0-4 of which are nitrogen, 0-2 of which are oxygen and 0-1 of which are sulfur;

3 R^{a} groups are present, 1-3 of which are selected from the group consisting of: hydrogen and halo, and 0-2 of which are selected from the group consisting of:

OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;

phenyl, heteroaryl, —O-phenyl and —O-heteroaryl, said phenyl and heteroaryl groups and portions being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH; NH₂ and CN;

and C₁₋₃alkyl and OC₁₋₃alkyl, the alkyl portions of which are optionally substituted with 1-3 halo atoms and 1-2 phenyl or heteroaryl groups, said phenyl and heteroaryl being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH, NH₂ and CN;

each R^{b} independently represents hydrogen, halo, C₁₋₃alkyl, haloC₁₋₃alkyl, OC₁₋₃alkyl, haloC₁₋₃alkoxy or OH, or two R^{b} groups may be combined to form a fused 5-6 membered ring, with two such rings being possible;

R^{c} represents —CO₂H or

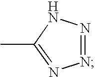

and each R^{d} independently represents H, halo, methyl, or methyl substituted with 1-3 halo atoms.

An aspect of the invention that is of interest relates to compounds of formula I or a pharmaceutically acceptable salt or solvate thereof wherein D represents a bond, an oxygen atom, —CH₂— or —CH₂CH₂—. Within this subset of the invention, all other variables are as originally defined with respect to formula I.

More particularly, an aspect of the invention that is of interest relates to compounds of formula I or a pharmaceutically acceptable salt or solvate thereof wherein D represents a bond. Within this subset of the invention, all other variables are as originally defined with respect to formula I.

Another aspect of the invention that is of interest relates to compounds of formula I or a pharmaceutically acceptable salt or solvate thereof wherein X represents a carbon atom. Within this subset of the invention, all other variables are as originally defined with respect to formula I.

Another aspect of the invention that is of interest relates to compounds of formula I or a pharmaceutically acceptable salt or solvate thereof wherein X represents a nitrogen atom. Within this subset of the invention, all other variables are as originally defined with respect to formula I.

Another aspect of the invention that is of interest relates to compounds of formula I or a pharmaceutically acceptable salt or solvate thereof wherein

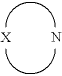

represents a 7 membered heterocyclic ring containing 1-2 nitrogen atoms. Within this subset of the invention, all other variables are as originally defined with respect to formula I.

Another aspect of the invention that is of interest relates to compounds of formula I or a pharmaceutically acceptable salt or solvate thereof wherein R^{c} represents a CO₂H group. Within this subset of the invention, all other variables are as originally defined with respect to formula I.

Another aspect of the invention that is of interest relates to compounds of formula I or a pharmaceutically acceptable salt or solvate thereof wherein each R^{d} represents a hydrogen or fluorine atom. Within this subset of the invention, all other variables are as originally defined with respect to formula I.

Another aspect of the invention that is of interest relates to compounds of formula I or a pharmaceutically acceptable salt or solvate thereof wherein each R^{b} is selected from a hydrogen atom and CH₃, or two R^{b} groups are taken in combination and represent a 5 membered ring, with one or two such rings being present. Within this subset of the invention, all other variables are as originally defined with respect to formula I.

More particularly, another aspect of the invention that is of interest relates to compounds of formula I or a pharmaceutically acceptable salt or solvate thereof wherein each R^{b} is selected from a hydrogen atom and CH₃. Within this subset of the invention, all other variables are as originally defined with respect to formula I.

Even more particularly, another aspect of the invention that is of interest relates to compounds of formula I or a pharmaceutically acceptable salt or solvate thereof wherein each R^{b} represents a hydrogen atom. Within this subset of the invention, all other variables are as originally defined with respect to formula I.

Even more particularly, another aspect of the invention that is of interest relates to compounds of formula I or a pharmaceutically acceptable salt or solvate thereof wherein 1-2 R^{b} groups represent methyl and the remainder represent hydrogen. Within this subset of the invention, all other variables are as originally defined with respect to formula I.

Another aspect of the invention that is of interest relates to compounds of formula I or a pharmaceutically acceptable salt or solvate thereof wherein two R^{b} groups are taken in combination and represent a 5 membered ring, with two such rings being present. Within this subset of the invention, all other variables are as originally defined with respect to formula I.

Another aspect of the invention that is of interest relates to compounds of formula I or a pharmaceutically acceptable salt or solvate thereof wherein B and B¹ are taken separately, such that B¹ represents a hydrogen atom and B represents a 6-10 membered aryl or a 5-10 membered heteroaryl group containing from 1-4 heteroatoms, 0-4 of which are nitrogen, 0-2 of which are oxygen and 0-1 of which is sulfur. Within this subset of the invention, all other variables are as originally defined with respect to formula I.

More particularly, another aspect of the invention that is of interest relates to compounds of formula I or a pharmaceutically acceptable salt or solvate thereof wherein B and B¹ are taken separately, B¹ represents H and B represents a 6-10 membered aryl group. Within this subset of the invention, all other variables are as originally defined with respect to formula I.

Even more particularly, another aspect of the invention that is of interest relates to compounds of formula I or a pharmaceutically acceptable salt or solvate thereof wherein B represents a naphthyl group. Within this subset of the invention, all other variables are as originally defined with respect to formula I.

Also more particularly, another aspect of the invention that is of interest relates to compounds of formula I or a pharmaceutically acceptable salt or solvate thereof wherein B and B¹ are taken separately, B¹ represents H and B represents a 5-10 membered heteroaryl group. Within this subset of the invention, all other variables are as originally defined with respect to formula I.

Another aspect of the invention that is of interest relates to compounds of formula I or a pharmaceutically acceptable salt or solvate thereof wherein B and B¹ are taken together and represent a spiro ring having 5-6 atoms. Within this subset of the invention, all other variables are as originally defined with respect to formula I.

More particularly, another aspect of the invention that is of interest relates to compounds of formula I or a pharmaceutically acceptable salt or solvate thereof wherein B and B¹ are taken together and represent a spiro ring having 5 or 6 atoms one of which is an oxygen atom. Within this subset of the invention, all other variables are as originally defined with respect to formula I.

Another aspect of the invention that is of interest relates to compounds of formula I or a pharmaceutically acceptable salt or solvate thereof wherein 2-3 R^{a} groups are selected from a hydrogen atom and halo. Within this subset of the invention, all other variables are as originally defined with respect to formula I.

More particularly, another aspect of the invention that is of interest relates to compounds of formula I wherein 0-1 R^{a} group is selected from the group consisting of:

OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;

phenyl, heteroaryl, —O-phenyl and —O-heteroaryl, said phenyl and heteroaryl groups and portions being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH, NH₂ and CN;

and the remaining R^{a} groups are hydrogen. Within this subset of the invention, all other variables are as originally defined with respect to formula I.

Even more particularly, another aspect of the invention that is of interest relates to compounds of formula I wherein 0-1 R^{a} group is selected from the group consisting of:

phenyl and heteroaryl, said phenyl and heteroaryl groups being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy OH, NH₂ and CN;

and C₁₋₃alkyl and OC₁₋₃alkyl, the alkyl portions of which are optionally substituted with 1-3 halo atoms and 1 phenyl or heteroaryl group, said phenyl and heteroaryl being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH, NH₂ and CN;

and the remaining R^{a} groups are hydrogen. Within this subset of the invention, all other variables are as originally defined with respect to formula I.

Also of more particular interest are compounds of formula I wherein: X represents a nitrogen atom, D represents a bond, B¹ is absent and B represents a 10 membered aryl or a 9-10 membered heteroaryl group containing from 1-4 heteroatoms, 0-4 of which are nitrogen, 0-2 of which are oxygen and 0-1 of which is sulfur, said group B being substituted with 3 R^{a} groups, one of which is OH and the remainder of which are hydrogen or halo atoms. Within this subset of the invention, all other variables are as originally defined with respect to formula I.

More particularly, an aspect of the invention that is of interest relates to compounds of formula I-A:

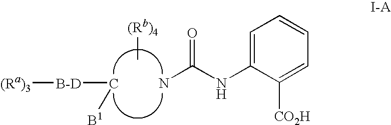

or a pharmaceutically acceptable salt or solvate thereof, wherein:

D represents a bond, an oxygen atom, —CH₂— or —CH₂CH₂—;

each R^{b} is selected from a hydrogen atom and CH₃ or two R^{b} groups are taken in combination and represent a 5 membered ring, with two such rings being present;

B and B¹ can be taken together or separately;

when B and B¹ are taken together, B and B¹ taken together represent a spiro ring containing 5-6 atoms, optionally containing 1 heteroatom or group selected from oxygen, sulfur, sulfinyl, sulfonyl and nitrogen, said spiro ring being optionally substituted with 1 oxo group, and optionally fused to a phenyl ring, said spiro or fused phenyl ring having 3 R^{a} groups,

and when B and B¹ are taken separately, B¹ represents hydrogen and

B represents a 6-10 membered aryl or a 5-10 membered heteroaryl group containing from 1-4 heteroatoms, 0-4 of which are nitrogen, 0-2 of which are oxygen and 0-1 of which are sulfur;

and 0-1 R^{a} groups are selected from

OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;

phenyl, heteroaryl, —O-phenyl and —O-heteroaryl, said phenyl and heteroaryl groups and portions being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy OH, NH₂ and CN;

and C₁₋₃alkyl and OC₁₋₃alkyl, the alkyl portions of which are optionally substituted with 1-3 halo atoms and 1 phenyl or heteroaryl group, said phenyl and heteroaryl being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH, NH₂ and CN,

and the remaining 2-3 R^{a} groups are selected from H and halo. Within this subset of the invention, all other variables are as originally defined with respect to formula I.

More particularly, an aspect of the invention that is of interest relates to compounds of formula I-B:

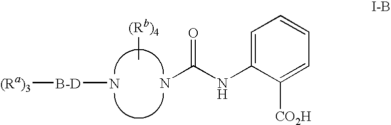

or a pharmaceutically acceptable salt or solvate thereof, wherein:

D represents a bond;

each R^{b} is selected from a hydrogen atom and CH₃ or two R^{b} groups are taken in combination and represent a 5 membered ring, with two such rings being present;

B represents a 6-10 membered aryl or a 5-10 membered heteroaryl group containing from 1-4 heteroatoms, 0-4 of which are nitrogen, 0-2 of which are oxygen and 0-1 of which are sulfur;

and 0-1 R^{a} groups are selected from

OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;

and the remaining 2-3 R^{a} groups are selected from H and halo. Within this subset of the invention, all other variables are as originally defined with respect to formula I.

Examples of compounds falling within the present invention are set forth below in Table 1:

TABLE 1

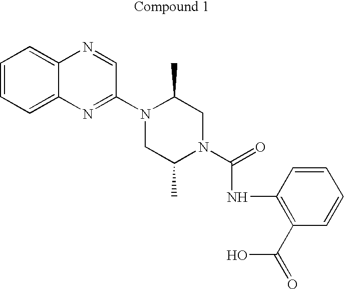

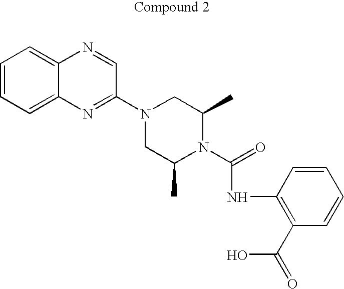

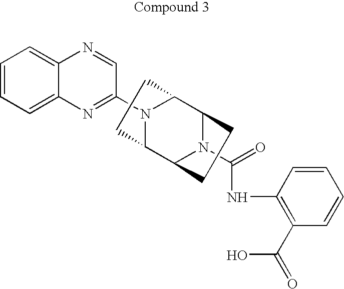

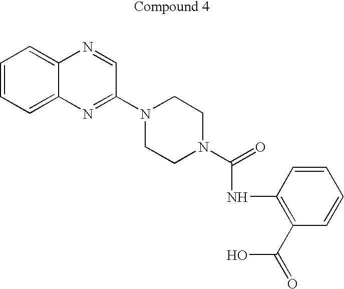

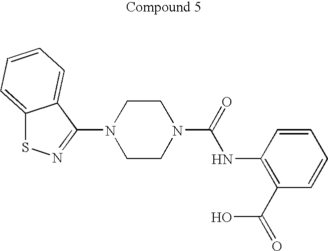

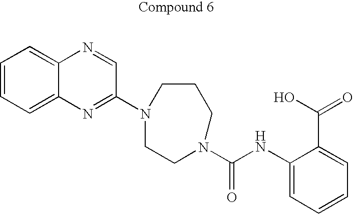

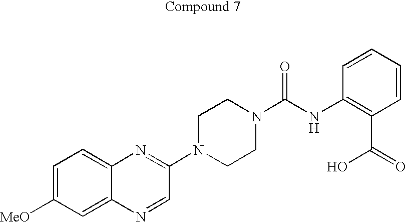

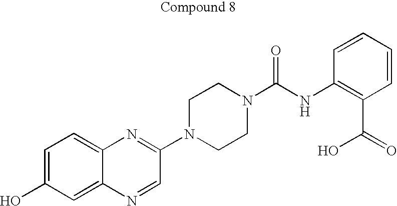

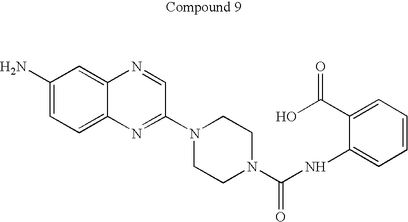

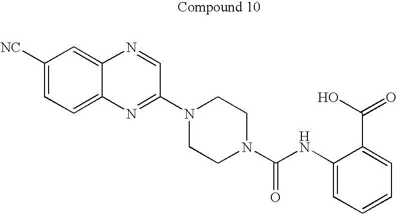

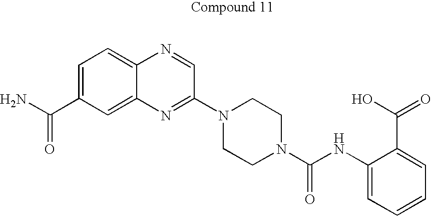

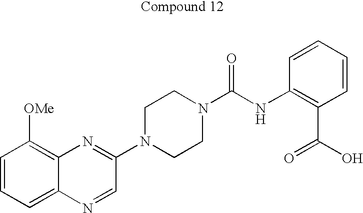

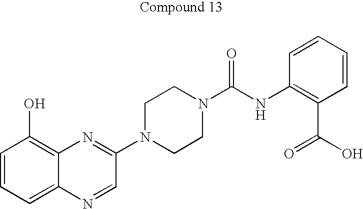

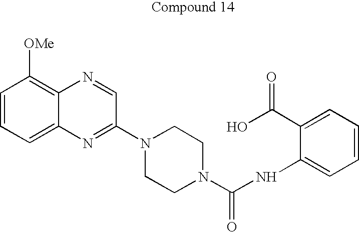

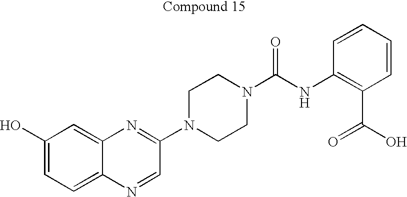

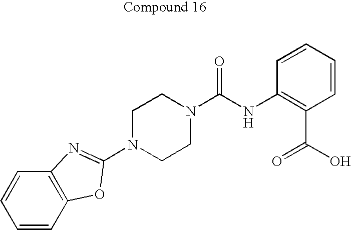

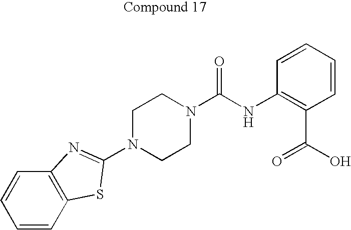

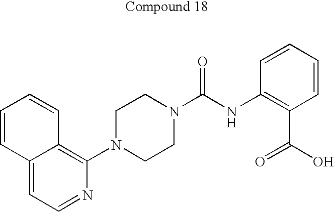

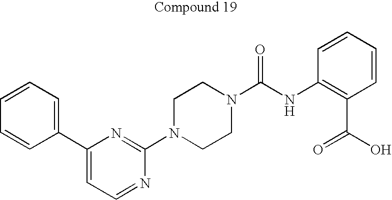

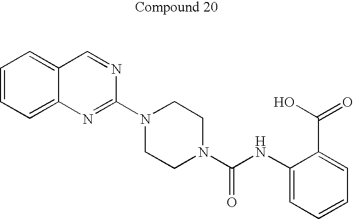

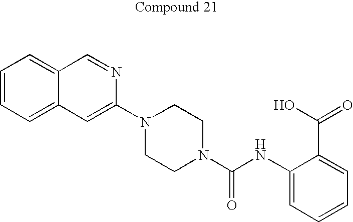

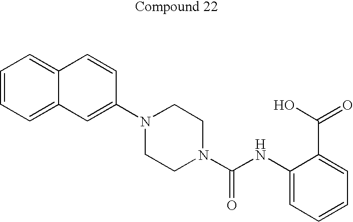

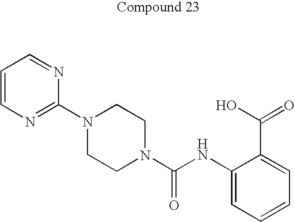

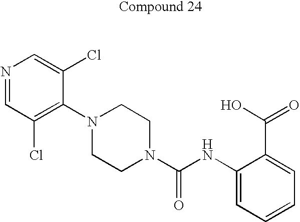

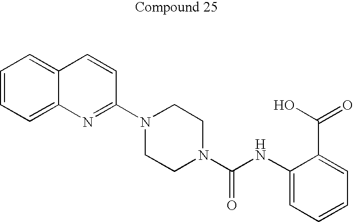

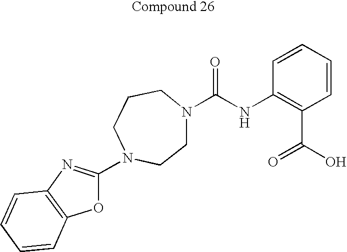

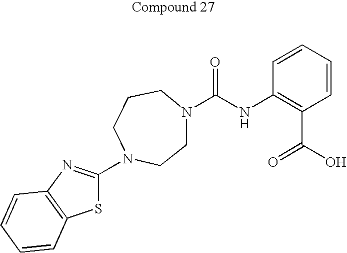

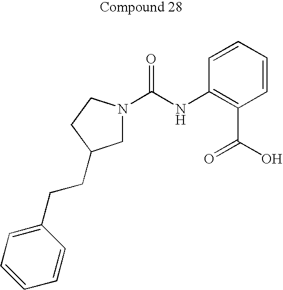

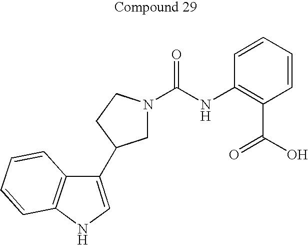

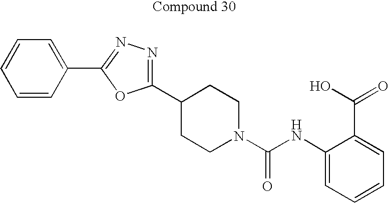

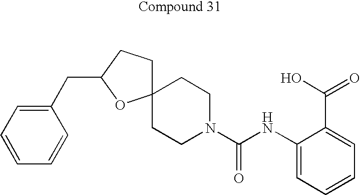

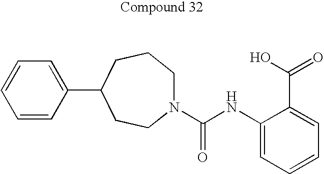

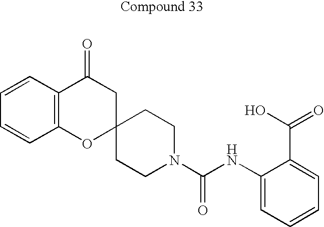

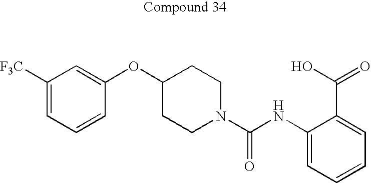

Pharmaceutically acceptable salts and solvates thereof are included as well.

Many of the compounds of formula I contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I can also be separated directly by chromatographic methods utilizing chiral stationary phases, which methods are well known in the art.

Alternatively, enantiomers of compounds of the general Formula I may be obtained by stereoselective synthesis using optically pure starting materials or reagents.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. The individual tautomers as well as mixtures thereof are included.

### Dosing Information

The dosages of compounds of formula I or a pharmaceutically acceptable salt or solvate thereof vary within wide limits. The specific dosage regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. Examples of suitable doses include about 0.1 mg, 1 mg, 2 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 400 mg, 500 mg, 600 mg, 750 mg, 900 mg, 1000 mg and the like. It is expected that the compounds described herein will be administered on a daily basis for a length of time appropriate to treat or prevent the medical condition relevant to the patient, including a course of therapy lasting months, years or the life of the patient.

### Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidine diones as well as those PPARγ agonists outside the thiazolidine dione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyancobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, farnesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower Kᵢ value) than the affinity at the CRTH2 receptor. Any compound that selectively interacts with DP according to these guidelines is deemed “DP selective”.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the following:

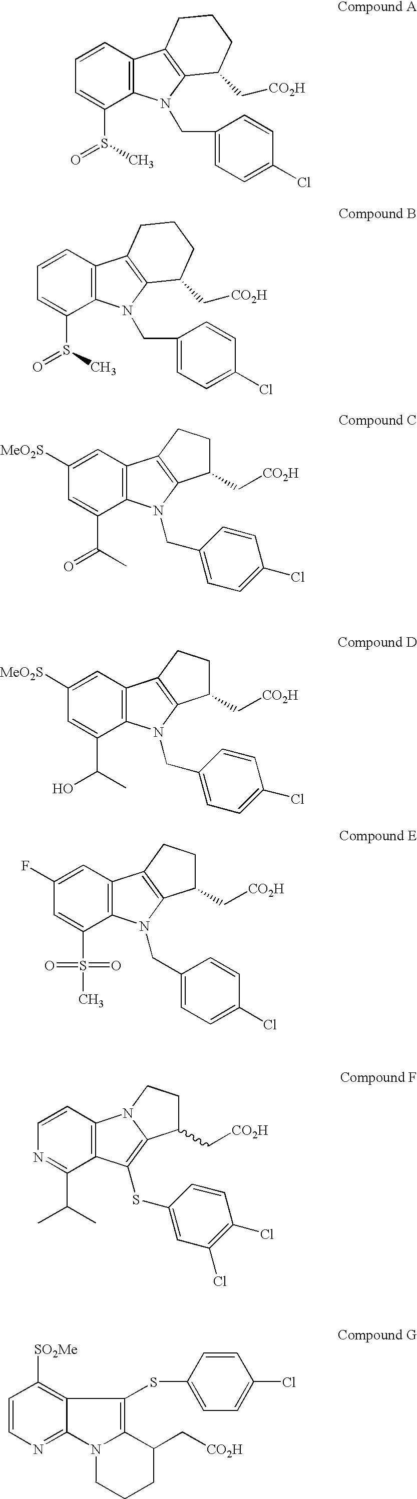

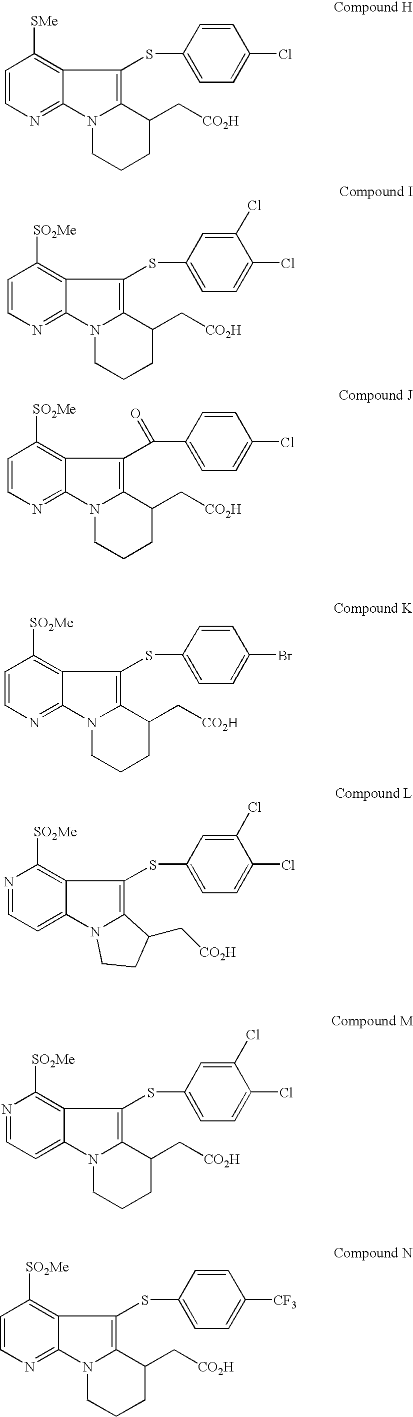

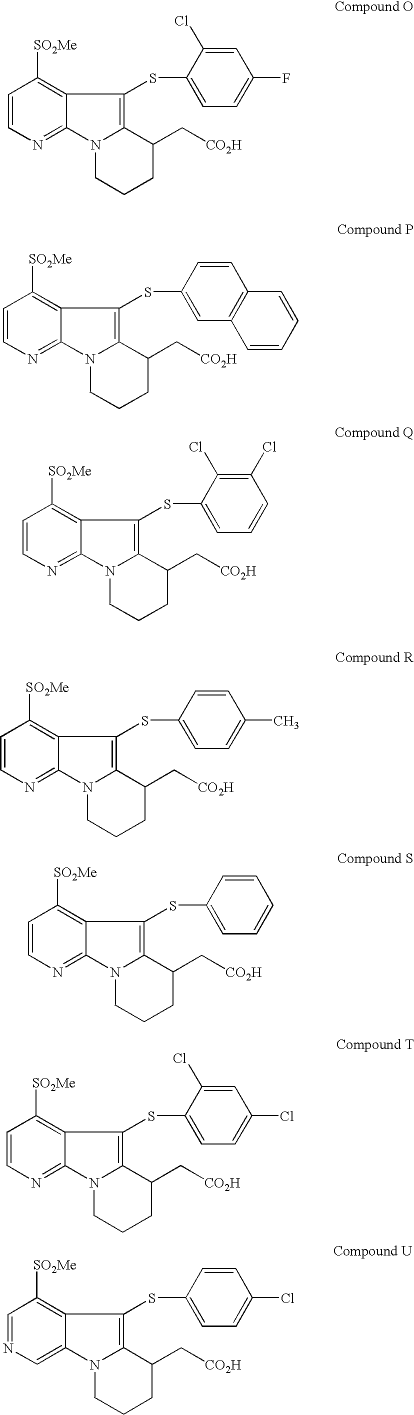

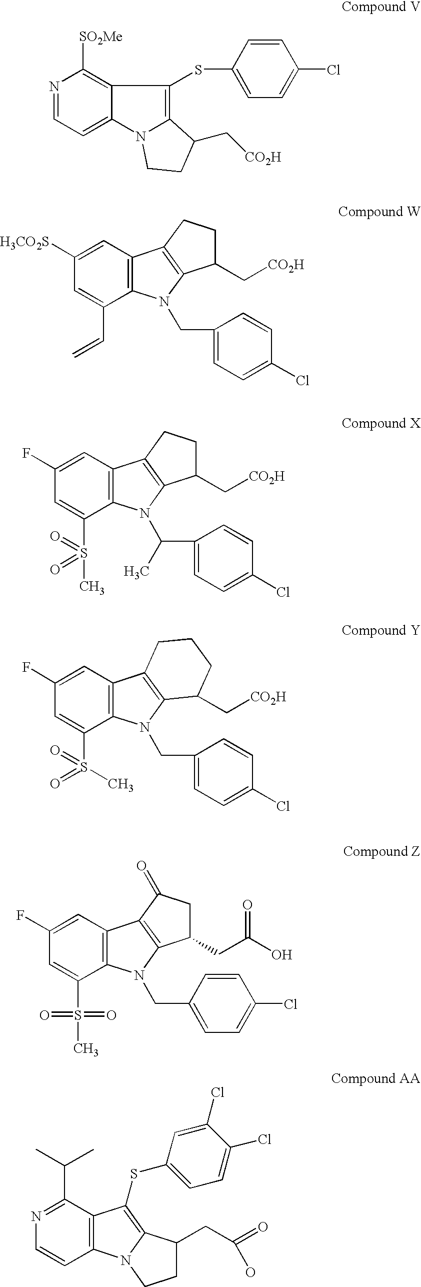

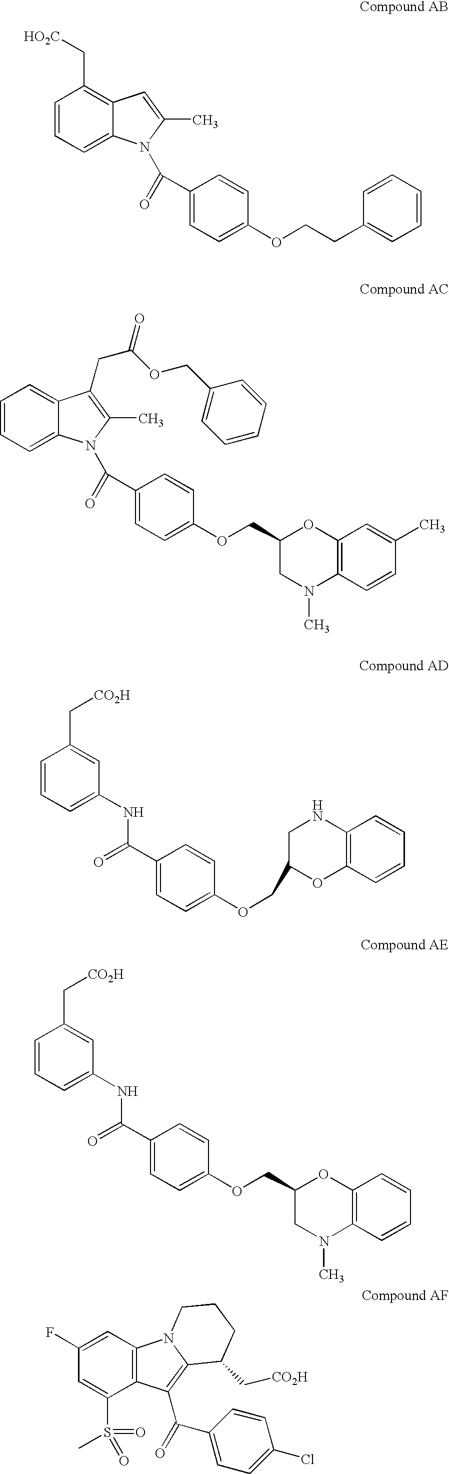

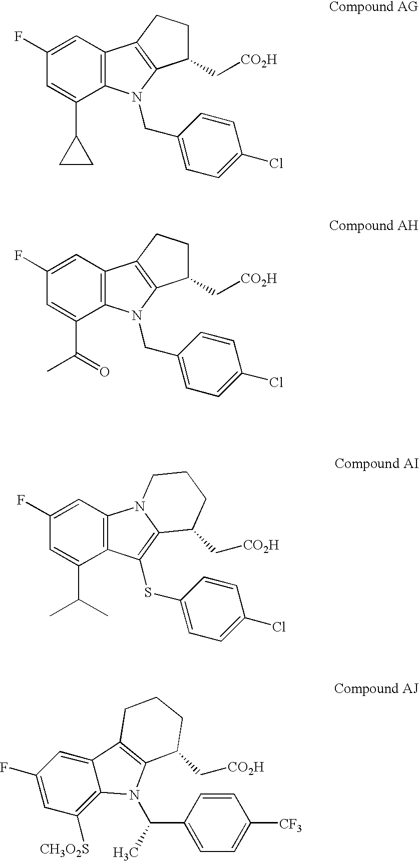

as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

### Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate. The compounds of the invention also include esters that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

The compounds used in the present invention can be administered via any conventional route of administration. The preferred route of administration is oral.

### Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, prevention or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

### Methods of Synthesis for Compounds of Formula I

Compounds of Formula I have been prepared by the following representative reaction schemes. It is understood that similar reagents, conditions or other synthetic approaches to these structure classes are conceivable to one skilled in the art of organic synthesis. Therefore these reaction schemes should not be construed as limiting the scope of the invention. All substituents are as defined above unless indicated otherwise.

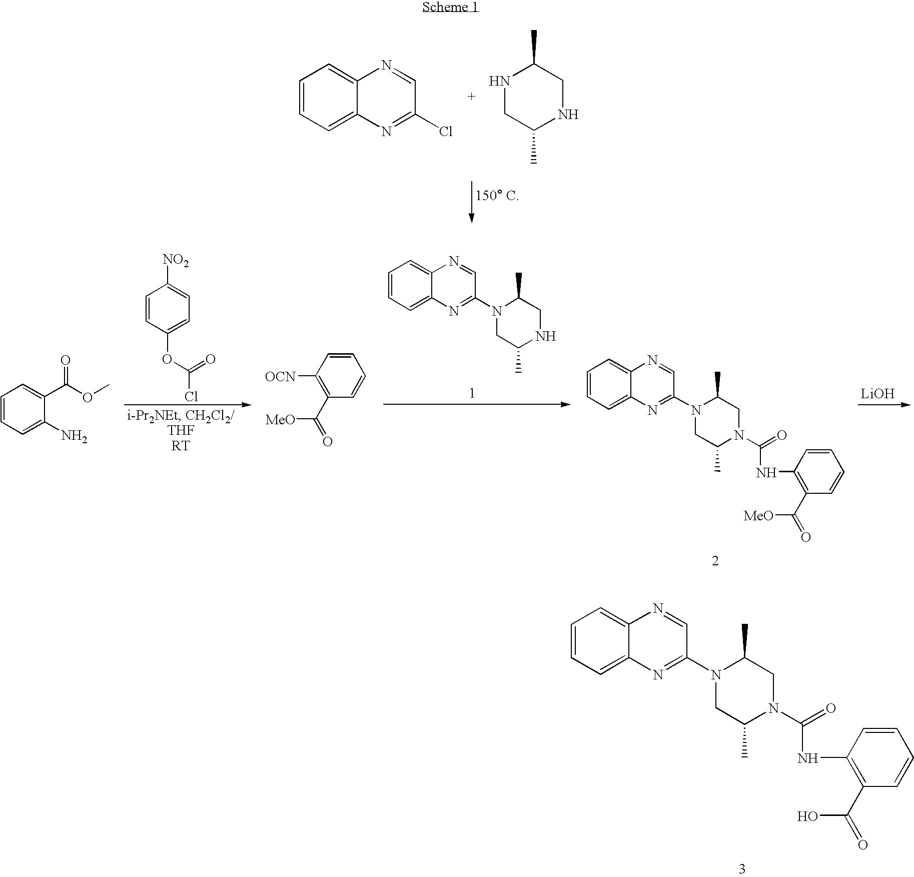

Compounds of Formula I can be prepared as illustrated in Scheme 1 by treatment of a chloroquinoxaline with a piperazine under thermal conditions to generate intermediates such as 1. Methyl anthranilate can be converted to its isocyanate, and then reacted with amine 1 to generate the urea 2. Saponification can generate acids such as 3, within this urea motif.

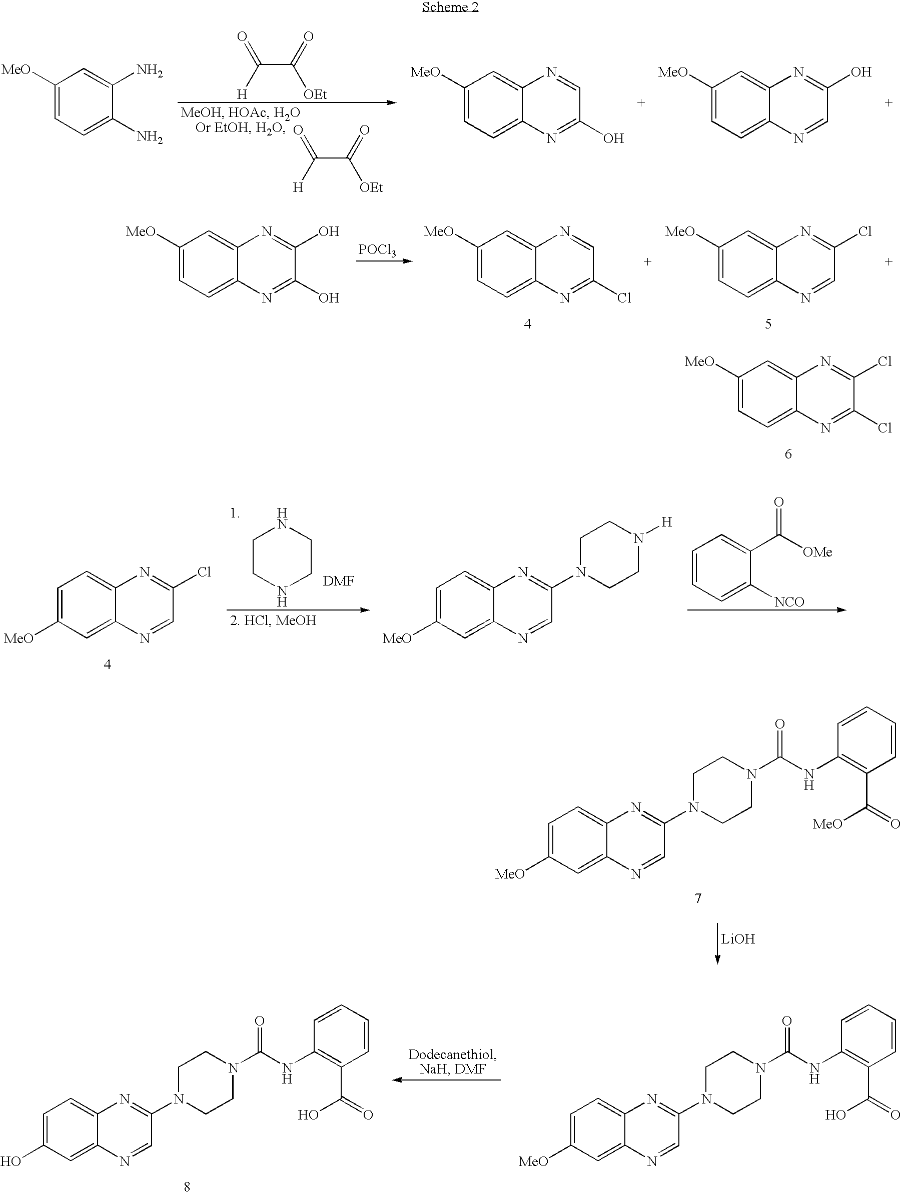

Compounds of Formula I can also be prepared as illustrated in Scheme 2, to access oxygen-substituted quinoxalines. Intermediates 4, 5 and 6, can be synthesized via condensation of the appropriately substituted diaminobenzene with ethyl glyoxylate, followed by chlorination of the hydroxyl quinoxalines. Thus the methoxy chloroquinoxaline 4, can be reacted with amines such as piperazine, and the resulting amine acylated with the isocyanate of methyl anthranilate to generate 7. The ester of 7 can be saponified, and the ether demethylated to provide compounds such as 8 by methods known to those skilled in the art.

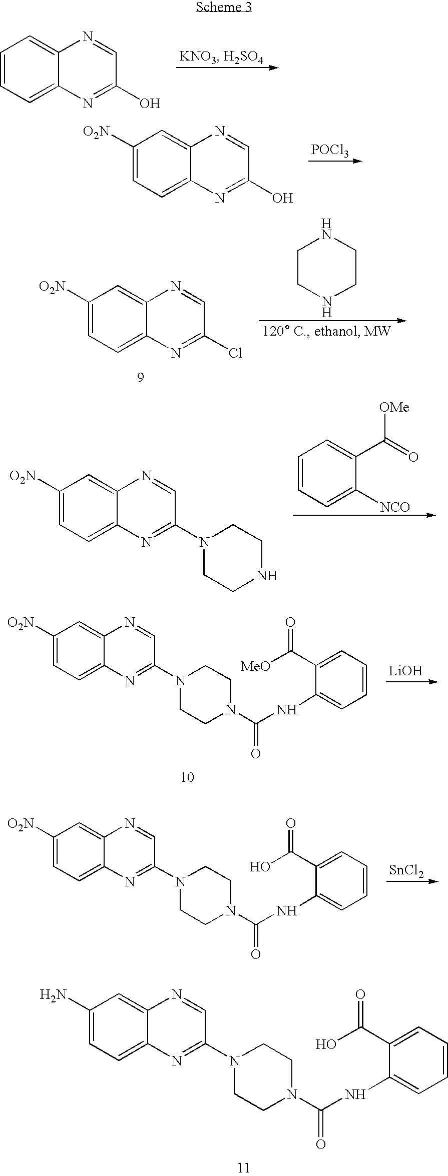

Shown in Scheme 3 is a preparation of nitrogen-substituted quinoxalines of Formula I. The hydroxyquinoxaline starting material can be nitrated, and the hydroxyl group chlorinated to generate intermediate 9. Chloride 9 can then be reacted with an amine such as piperazine, and the resulting amine acylated with the isocyanate of methyl anthranilate to provide 10. Saponification followed by reduction of the nitro moiety can provide products such as 11.

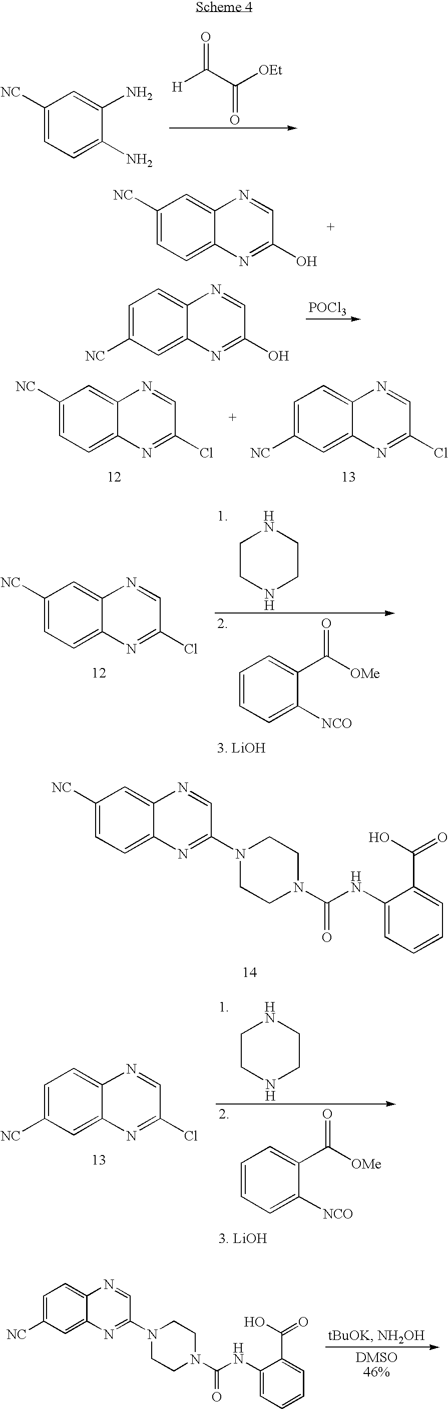

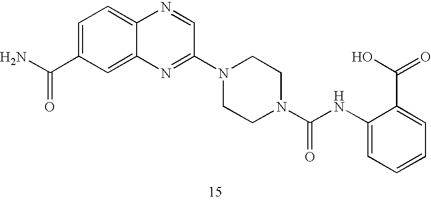

Compounds of Formula I can also be prepared as illustrated in Scheme 4. Intermediates 12 and 13, can be synthesized via condensation of the appropriately substituted diaminobenzene with ethyl glyoxylate, followed by chlorination of the hydroxyl quinoxalines. Thus the cyano chloroquinoxaline 12, can be reacted with amines such as piperazine, and the resulting amine acylated with the isocyanate of methyl anthranilate to generate compounds such as 14, after saponification. Similarly, the cyano chloroquinoxaline 13, can be converted to the regioisomer of nitrile 14, followed by generation of a primary carboxamide, such as 15.

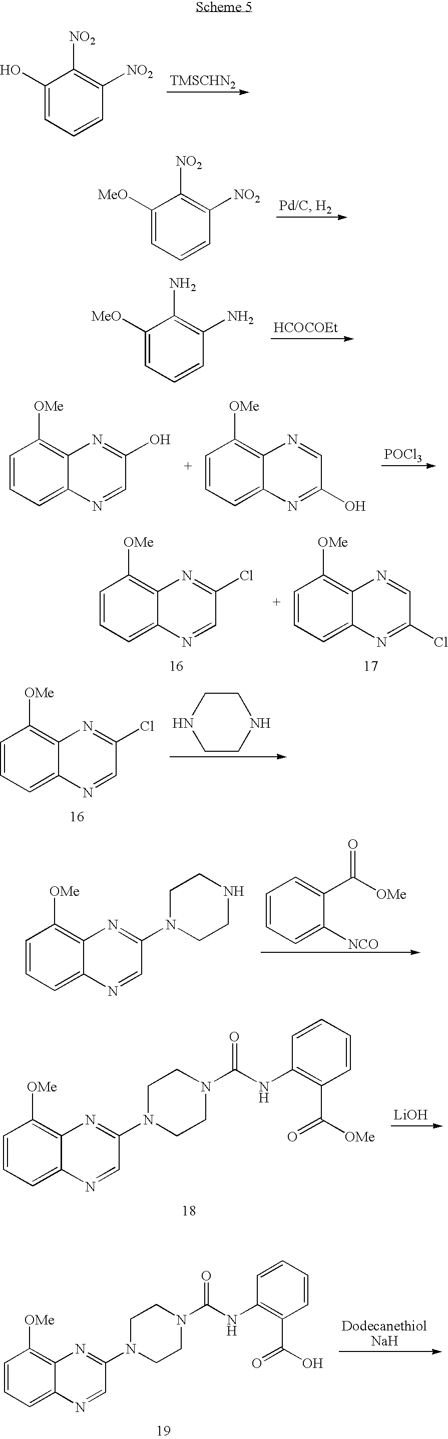

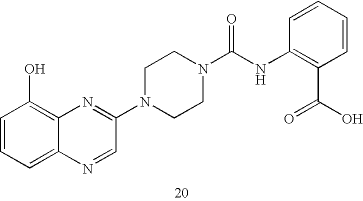

Other regioisomeric oxygenated quinoxaline derivatives of Formula I may be obtained following the chemistry illustrated in Scheme 5. Intermediates 16 and 17 can be accessed from dinitrophenol, by first methyl ether formation, reduction to the diaminobenzene, followed by condensation with ethyl glyoxylate, and then chlorination. Thus the methoxy chloroquinoxaline 16, can be reacted with amines such as piperazine, and the resulting amine acylated with the isocyanate of methyl anthranilate to generate 18. The ester of 18 can be saponified to 19, and the ether demethylated to form compounds such as 20.

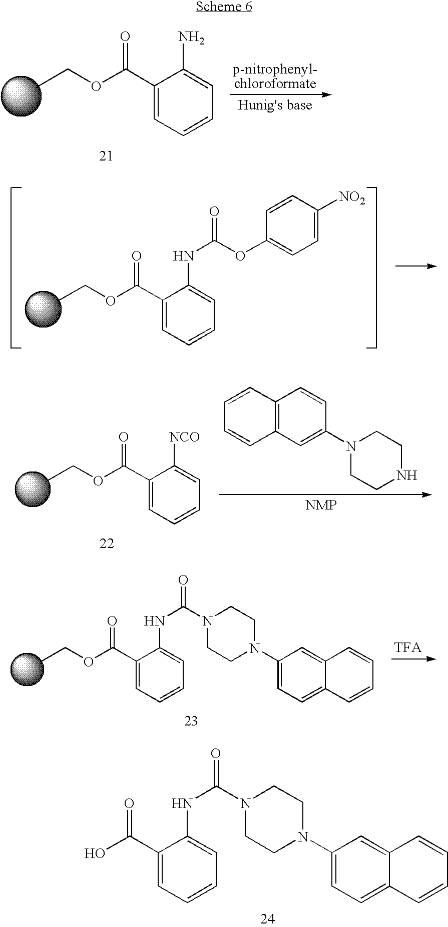

Scheme 6 outlines a solid phase synthesis strategy used to create compounds of the Formula I. Resin-supported anthranilate 21 can be converted to the isocyanate 22. This solid phase electrophile 22 can be reacted with a variety of amines to generate ureas such as naphthyl intermediate 23. Cleavage of the product urea from the resin using acidic conditions known to those skilled in the art, provides compounds such as 24.

The various organic group transformations and protecting groups utilized herein can be performed by a number of procedures other than those described above. References for other synthetic procedures that can be utilized for the preparation of intermediates or compounds disclosed herein can be found in, for example, M. B. Smith, J. March Advanced Organic Chemistry, 5^{th} Edition, Wiley-Interscience (2001); R.C. Larock Comprehensive Organic Transformations, A Guide to Functional Group Preparations, 2^{nd} Edition, VCH Publishers, Inc. (1999); T.L. Gilchrist Heterocyclic Chemistry, 3^{rd} Edition, Addison Wesley Longman Ltd. (1997); J. A. Joule, K. Mills, G. F. Smith Heterocyclic Chemistry, 3^{rd} Edition, Stanley Thornes Ltd. (1998); G. R. Newkome, W.W. Paudler Contempory Heterocyclic Chemistry, John Wiley and Sons (1982); or Wuts, P. G. M.; Greene, T. W.; Protective Groups in Organic Synthesis, 3^{rd} Edition, John Wiley and Sons, (1999), all six incorporated herein by reference in their entirety.

### REPRESENTATIVE EXAMPLES

The following examples are provided to more fully illustrate the present invention, and shall not be construed as limiting the scope in any manner. Unless stated otherwise:

(i) all operations were carried out at room or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (¹H NMR) spectrometry, and the purity was assured by at least one of the following techniques: TLC or HPLC;

(vi) 1H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid−acetonitrile, A=0.1% formic acid−water), Waters XTerra C18-3.5 um-50×3.0 mmID and diode array detection;

(viii) automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA), or alternatively in a library setting, automated purification of compounds by preparative reverse phase HPLC was performed on an Agilent system using an Agilent Combi, SB-C18 column (100×21.2 mm i.d.) eluting at 10 mL/min over 18 minutes with a step gradient elution of xx % acetonitrile in water (containing 0.1% TFA) as follows (3 min at 20%, then 14 min at 90%, then 1 min at 10%), fractions collected based upon mass along with diode array and ELSD detection;

(ix) column chromatography was carried out on a Biotage cartridge system;

(x) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar);

(xi) definitions of acronyms are as follows:

THF is tetrahydrofuran;

DMF is dimethylformamide;

NMP is N-methyl-2-pyrrolidinone;

TFA is trifluoroacetic acid;

DMAP is 4-dimethyl amino pyridine

DMSO is dimethyl sulfoxide

### Acylation of Resin

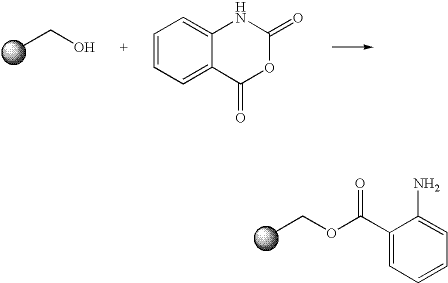

Wang resin (1.21 mmol/g, 200 mg, 0.24 mmol) was swollen in DMF (1 mL), and the anhydride (195 mg, 1.2 mmol) was added as a solution in DMF (1 mL), followed by DMAP (15 mg, 0.12 mmol). The reaction mixture was heated in a sealed tube at 80° C. for 3 h, with occasional agitation, cooled to room temperature, the resin filtered, and washed thrice each with DMF, methylene chloride, and hexanes.

### Example 1

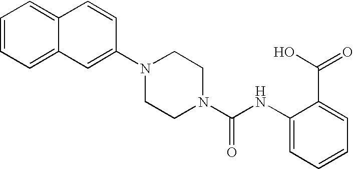

The acylated resin shown in Scheme 6 (200 mg, 0.2 mmol) was tared into 36 SPE cartridges. A solution of para-nitrophenyl chloroformate (16.2 g, 80 mmol) was dissolved in 50% methylene chloride—THF (150 mL) and chilled to 0° C. Hunig's base (14 mL, 80 mmol) was slowly added, and the flask was allowed to warm to room temperature. The resin was pre-swollen in each cartridge with THF (1 mL) and briefly aged. The para-nitrophenyl chloroformate solution prepared above (4 mL, 0.5M, 2 mmol, 10 equivalents) was added to each cartridge, and the reaction cartridges rotated overnight. The cartridges were then drained, washed twice with dry THF (3 mL), and the resin in one cartridge (for example) then treated with a solution of N-2-naphthylpiperizine (1 mmol) in NMP (3 mL). The reaction cartridge was rotated overnight, drained, (any cartridges with insoluble material were individually washed with glacial acetic acid), the resulting resin was washed thrice each with DMF, THF and then methylene chloride. The resin was then treated with TFA (1.5 mL), aged 1 h, the cartridge drained and collected, the filtered resin washed with TFA (0.75 mL), and the combined TFA fractions concentrated in vacuo. LCMS analysis was conducted by re-dissolving in methanol. This methanol solution of the crude product was purified on an automated Agilent preparative HPLC system. LCMS m/z 375 (M⁺).

### Examples 2-20

The following compounds were prepared under conditions similar to those described in Example 1 above and illustrated in Scheme 6.

| EXAMPLE | | LCMS (m/z) |
|---|---|---|
| 2 | 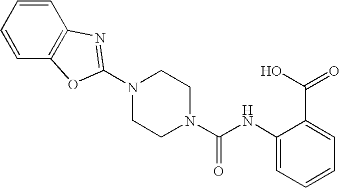 | 367 (M + 1) |
| 3 | 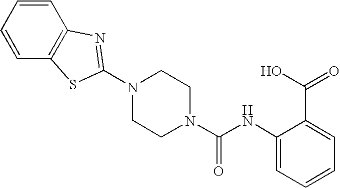 | 383 (M + 1) |
| 4 | 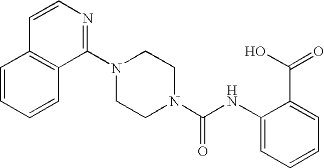 | 377 (M + 1) |
| 5 | 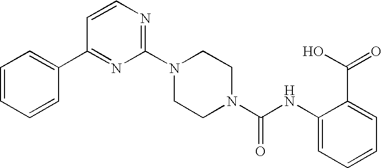 | 404 (M + 1) |
| 6 | 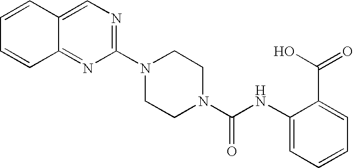 | 378 (M + 1) |
| 7 | 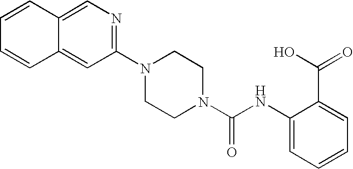 | 377 (M + 1) |
| 8 | 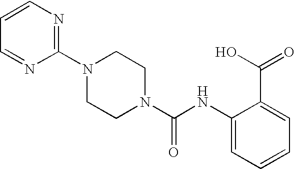 | 328 (M + 1) |
| 9 | 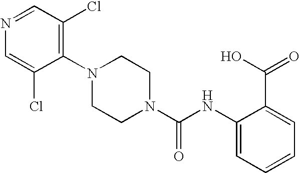 | 395 (M + 1) 397 (M + 3) |
| 10 | 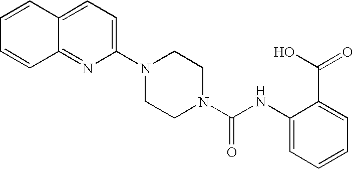 | 377 (M + 1) |
| 11 | 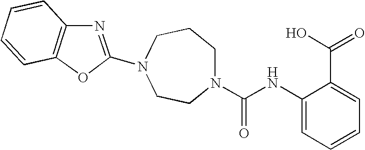 | 381 (M + 1) |
| 12 | 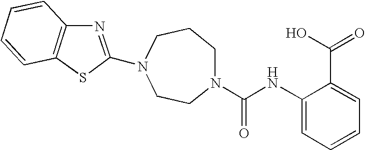 | 397 (M + 1) |
| 13 | 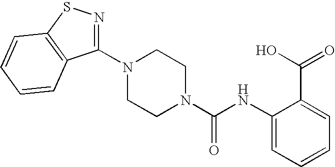 | 383 (M + 1) |
| 14 | 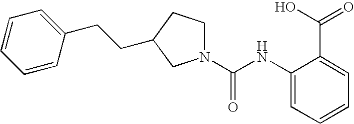 | 339 (M + 1) |
| 15 | 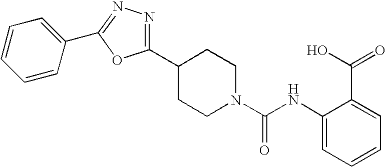 | 393 (M + 1) |
| 16 | 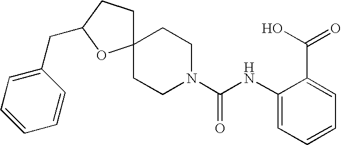 | 395 (M + 1) |
| 17 | 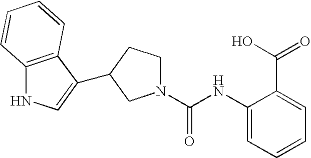 | 350 (M + 1) |
| 18 | 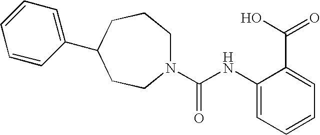 | 339 (M + 1) |
| 19 | 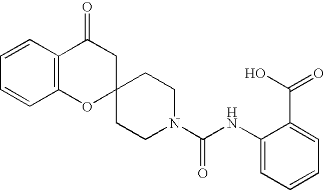 | 381 (M + 1) |
| 20 | 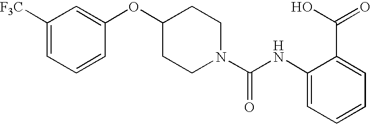 | 409 (M + 1) |

### NMR Data for Selected Examples:

### Example 13

¹H NMR (acetone-d₆, 500 MHz) δ 8.63 (1H, d), 8.15 (2H, m), 8.01 (1H, d), 7.53 (3H, m), 7.04 (2H, m), 3.81 (4H, m), 3.61 (4H, m).

### Example 21

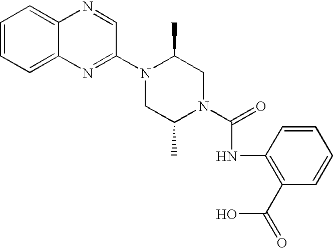

A mixture of chloroquinoxaline (165 mg, 1 mmol) and dimethylpiperazine (570 mg, 5 mmol) in 2 mL of DMF was heated in microwave (Pmax=300 W) for 10 min. Purification by reverse phase HPLC afforded the product as a pale brown solid. A solution of this intermediate (50 mg, 0.14 mmol) in 4 mL of dichloromethane was treated with a stock solution of the isocyanate of methyl anthranilate (0.2M, 2 mL, 0.40 mmol); prepared by mixing methyl anthranilate (907 mg, 6 mmol), diisopropylethylamine (3.1 g, 4.2 mL, 24 mmol) and 30 mL of dichloromethane with p-nitrophenylchloroformate (1.21 g, 6 mmol) at 0° C., with warming to room temperature overnight, to provide a bright yellow isocyanate stock solution (˜0.2 M). The resulting reaction mixture was stirred at room temperature for 3 h, and then concentrated in vacuo. The residue was dissolved in DMSO and purified by reverse phase HPLC to afford the product as a yellow oil. This methyl ester was dissolved in (3:1:1) THF:methanol:water, and treated with lithium hydroxide (1 mL, 1N in water). The mixture was stirred for 1 h and then concentrated in vacuo. The residue was washed with chloroform and then acidified with concentrated HCl until pH=3. The mixture was extracted with 30% isopropanol in chloroform. The organic phase was washed with water, dried with sodium sulfate. After the removal of solvent, the product was obtained as a yellow solid. ¹H NMR (acetone-d₆, 500 MHz) δ 8.86 (1H, s), 8.42 (1H, d), 7.98 (1H, d), 7.84 (1H, d), 7.57 (2H, m), 7.55 (1H, t), 7.42 (1H, t), 7.03 (1H, t), 4.96 (1H, bs), 4.48 (1H, bs), 4.33 (1H, d), 4.13 (1H, bs), 3.86 (1H, d), 3.78 (1H, m), 1.26 (6H, m); LCMS m/z 406 (M+1).

### EXAMPLES 22-25

The following compounds were prepared under conditions similar to those described in Example 21 above and illustrated in Scheme 1.

| | | LCMS |
|---|---|---|
| EXAMPLE | | (m/z) |
| 22 | 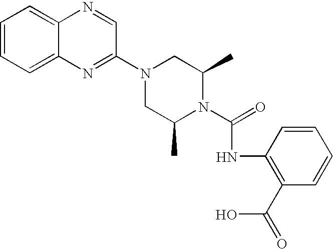 | 406 (M + 1) |
| 23 | 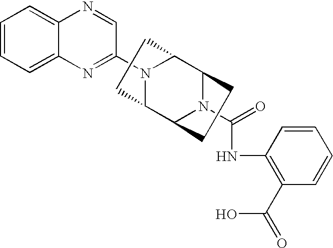 | 430 (M + 1) |
| 24 | 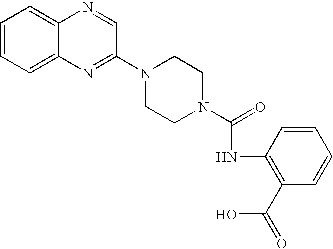 | 378 (M + 1) |
| 25 | 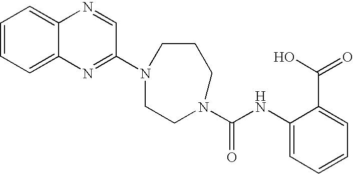 | 392 (M + 1) |

### NMR Data for Selected Examples:

### Example 22

¹H NMR (acetone-d₆, 500 MHz) δ 8.89 (1H, s), 8.71 (1H, d), 8.10 (1H, d), 7.87 (1H, t), 7.67 (3H, m), 7.32 (1H, t), 7.05 (1H, t), 4.89 (1H, d), 4.71 (1H, d), 4.55 (1H, m), 3.67 (1H, m), 3.37 (2H, m), 1.52 (3H, d), 1.43 (3H, d).

### Example 23

¹H NMR (acetone-d₆, 500 MHz) δ 8.79 (1H, s), 8.67 (1H, d), 8.11 (1H, dd), 7.85 (1H, d), 7.62 (3H, m), 7.42 (1H, m), 7.07 (1H, t), 5.07 (2H, s), 4.56 (2H, s), 1.96 (4H, m), 1.83 (4H, m).

### Example 24

¹H NMR (DMSO-d₆, 500 MHz) δ 8.85 (1H, s), 8.41 (1H, d), 8.12 (1H, d), 7.96 (1H, dd), 7.83 (1H, d), 7.62 (2H, m), 7.52 (1H, t), 7.41 (1H, t), 7.01 (1H, t), 6.92 (1H, d), 3.90 (2H, m), 3.67 (2H, m), 1.96 (2H, m), 1.83 (2H, m).

### Example 25

1H NMR (DMSO-d₆, 500 MHz) δ 11.1 (1H, br), 10.9 (1H, s), 8.71 (1H, s), 8.31 (1H, d), 7.90 (1H, d), 7.76 (1H, d), 7.53 (2H, s), 7.42 (1H, t), 7.33 (1H, m), 4.03 (2H, m), 3.84 (2H, m), 3.60 (2H, m), 3.25 (2H, m), 2.02 (2H, m).

### Example 26

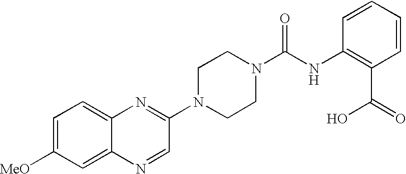

To a suspension of diaminomethoxybenzene (2.1 g, 10 mmol) in 25 mL of water and 10 mL of ethanol was added sodium bicarbonate (1.7 g, 20 mmol) to neutralize. To the resulting mixture was added ethyl glyoxylate (2.04 g, 2 μL, 11 mmol) and the mixture was then under reflux for 2 h. The mixture was cooled and filtered. The solid was dissolved in DMSO and purified by RP-HPLC to afford a mixture of regioisomeric alcohols (4:1:1) as shown in Scheme 2. To this mixture of alcohols (1.76 g, 10.0 mmol) was added 40 mL of POCl₃. The resulting mixture was under reflux for 1 h. The mixture was concentrated by distillation off the solvent. The residue was poured onto ice and basified with sodium carbonate. The mixture was extracted with ethyl acetate. The organic layer was washed with brine, dried with sodium sulfate and concentrated in vacuo. The residue was purified by flash chromatography eluting with 5% ethyl acetate in hexane to separate the single isomeric chlorides. A mixture of a single isomeric chloride (220 mg, 1.13 mmol), piperazine (440 mg, 5.1 mmol) and 4 mL of butanol was heated at 150° C. in microwave for 15 min, then 170° C. in microwave for additional 15 min. The mixture was purified by RP-HPLC to give the product as a yellow oil. Following the same procedure as described in EXAMPLE 21 for the preparation of the urea and the subsequent hydrolysis, the desired methyl ether product was obtained as a yellow solid. ¹H NMR (acetone-d₆, 500 MHz) δ 8.80 (1H, m), 8.42 (1H, d), 7.96 (1H, d), 7.58 (2H, m), 7.29 (2H, d), 7.03 (1H, t), 3.86 (3H, s), 3.81 (4H, m), 3.65 (4H, m); LCMS m/z 408 (M+1).

### Example 27

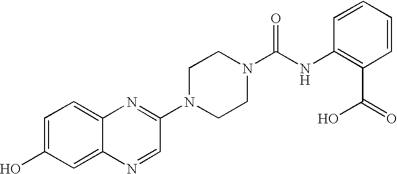

To sodium hydride (10 mg, 0.25 mmol, 60%) in 3 mL of DMF at 0° C. was added dodecanethiol (25 mg, 0.25 mmol). The mixture was warmed to room temperature and stirred for 15 min. To this mixture was added a solution of EXAMPLE 26 (10 mg, 0.025 mmol) in 3 mL of DMF. The mixture was heated at 120° C. for 6 h. The mixture was filtered and purified by Gilson to afford the desired hydroxyl product as a brown oil. ¹H NMR (acetone-d₆, 500 MHz) δ 8.80 (1H, m), 8.66 (1H, d), 8.00 (1H, s), 7.59 (2H, m), 7.28 (2H, d), 7.05 (1H, t), 3.90 (m, 4H), 3.78 (m, 4H); LCMS m/z 394 (M+1).

EXAMPLE 28 was prepared from the regioisomeric intermediates generated in EXAMPLE 26, following the same reaction conditions described in EXAMPLES 26 and 27 above.

| EXAMPLE | | LCMS (m/z) |
|---|---|---|
| 28 | 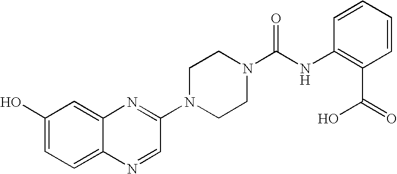 | 394 (M + 1) |

### Example 29

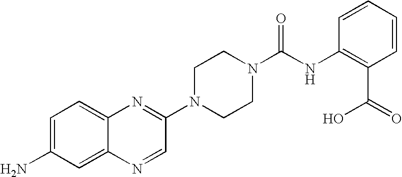

Powered potassium nitrate was added rapidly at 0° C. to a stirred solution of hydroxyquinoxaline (4.38 g, 30 mmol) in 50 mL of concentrated sulfuric acid. After 30 min at 0° C., and then room temperature for another 2 h, the mixture was slowly poured into crushed ice at 0° C. (˜250 mL). The precipitate was washed with 15 mL of water. Crystallization from acetic acid (200 mL) gave the nitro product as a white solid. To this hydroxy intermediate (502 mg, 2.63 mmol) was added 8 mL of POCl₃. The resulting mixture was heated at 110° C. for 2 h. The mixture was concentrated by distillation of the solvent. The residue was poured onto ice and basified with sodium carbonate until pH>8. The mixture was extracted with ethyl acetate (200 μL×10). The organic layer was washed with brine, dried with sodium sulfate and concentrated in vacuo to give the nitrochloride product as a dark pink solid. A mixture of this nitrochloride (385 mg, 1.84 mmol), piperazine (600 mg) and 2 mL of ethanol was heated at 120° C. in a microwave for 10 min (Pₘₐₓ=100 W). The mixture was concentrated and dissolved in DMSO before it was purified by RP-HPLC to give the product as a dark brown oil. Following the same procedure as described in EXAMPLE 21 for the preparation of the urea and the subsequent hydrolysis, the desired nitro product was obtained as a yellow solid. To a solution of this nitro intermediate (20 mg, 0.047 mmol) in 1 mL of DMF/water (10:1) was added tin(II) chloride hydrate (23 mg, 0.12 mmol) at room temperature. The mixture was stirred for 16 h, and then quenched with saturated sodium bicarbonate solution. The mixture was filtered through celite and washed with 30% isopropanol/chloroform. The filtrate was concentrated and purified by Gilson to give the desired product as a reddish brown solid. ¹H NMR (acetone-d₆, 500 MHz) δ 8.96 (1H, s), 8.64 (1H, d), 8.09 (1H, dd), 7.99 (1H, d), 7.83 (1H, d), 7.72 (1H, dd), 7.58 (1H, t), 7.06 (1H, t), 4.09 (m, 4H), 3.82 (m, 4H); LCMS m/z 393 (M+1).

### Example 30

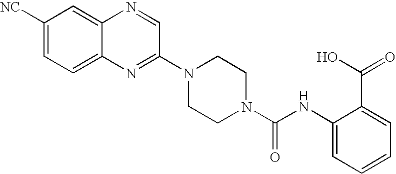

To a suspension of diaminocyanobenzene (1 g, 7.5 mmol) in 10 mL of ethanol was added sodium bicarbonate to neutralize the mixture. To the resulting mixture was added ethyl glyoxylate (1.7 g, 1.64 mL, 8.3 mmol, 50% in toluene), and the mixture was then stirred under reflux for 2 h. The mixture was cooled, filtered, and the solid was dissolved in DMSO, and purified by RP-HPLC to afford the product as a mixture of cyano regioisomers. To this mixture (1.15 g, 6.7 mmol) was added 15 mL of POCl₃. The resulting mixture was stirred under reflux for 1 h. The mixture was concentrated by distillation of the solvent. The residue was poured onto ice, and basified with sodium carbonate, and the mixture was extracted with ethyl acetate. The organic layer was washed with brine, dried with sodium sulfate, and concentrated in vacuo. A small fraction of the residue was purified by HPLC-AD column (4.6×250 mm Chiralpak AD, 2.1 mL/min, 1500 psi, 10% methanol/CO₂) eluting with 5% isopropanol in heptane (40 min/run) to separate the single regioisomeric chlorides. Following the same procedure as described in EXAMPLE 21 for the preparation of the urea and the subsequent hydrolysis, the desired nitrile product was obtained as a yellow oil. ¹H NMR (DMSO-d₆, 500 MHz) δ 10.97 (1H, s), 8.96 (1H, s), 8.41 (1H, d), 8.38 (1H, s), 7.96 (1H, d), 7.88 (1H, d), 7.68 (1H, d), 7.54 (1H, t), 7.03 (1H, t), 3.99 (m, 4H), 3.69 (m, 4H); LCMS m/z 403 (M+1).

### Example 31

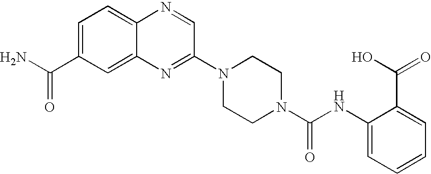

To a solution of hydroxylamine hydrochloride (17 mg, 0.25 mmol) in 2 mL of DMSO was added potassium t-butoxide (28 mg, 0.25 mmol). After 30 min, to this mixture was added the regioisomeric cyanide of EXAMPLE 30 (5 mg, 0.012 mmol). After 14 h, the mixture was directly purified by Gilson to give the primary carboxamide product as a yellow oil. ¹H NMR (DMSO-d₆, 500 MHz) δ 8.91 (1H, s), 8.41 (1H, d), 8.17 (2H, d), 7.96 (1H, d), 7.87 (2H, q), 7.50 (2H, m), 7.01 (1H, t), 3.92 (m, 4H), 3.68 (m, 4H); LCMS m/z 419 (M−1).

### Example 32

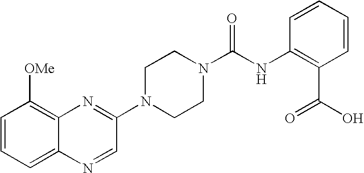

To a solution of dinitrophenol (1 g, 5.43 mmol) in 15 mL of methanol was added trimethylsilyl diazomethane (20 mL, 40 mmol, 2 M in hexane). After aging the reaction mixture for 2 h at room temperature, a few drops of acetic acid was added until gas evolution stopped. The removal of solvent gave the methyl ether as a crude product which was submitted to the subsequent reduction. To a solution of the dinitro intermediate in 30 mL of methanol was added Pd/C (100 mg). The slurry was stirred under 1 atm of hydrogen overnight. The mixture was filtered through celite and washed with acetone (50 mL). The red reaction mixture was concentrated to give the diamino methyl ether as a dark red oil. The conversion of this diamine to the subsequent quinoxaline hydroxy and chloride regioisomers, followed by conversion to the desired final product urea, followed a similar procedure as described in EXAMPLE 30. ¹H NMR (DMSO-d₆, 500 MHz) δ 10.96 (1H, s), 8.72 (1H, s), 8.41 (1H, d), 8.11 (1H, dd), 7.96 (1H, dd), 7.53 (2H, m), 7.18 (1H, d), 7.03 (1H, t), 6.92 (1H, dd), 6.88 (1H, d), 3.92 (3H, s), 3.87 (m, 4H), 3.66 (m, 4H); LCMS m/z 408 (M+1).

EXAMPLE 33 was prepared from the regioisomeric intermediates generated in EXAMPLE 32, following the same reaction conditions described in EXAMPLE 32 above.

| | | LCMS |
|---|---|---|
| EXAMPLE | | (m/z) |
| 33 | 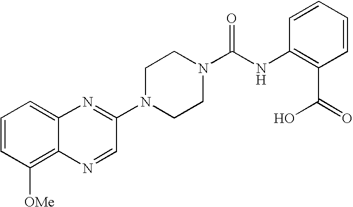 | 408 (M + 1) |

### NMR Data for Selected Examples:

### Example 33

¹H NMR (DMSO-d₆, 500 MHz) δ 10.96 (1H, s), 8.82 (1H, s), 8.41 (1H, d), 7.96 (1H, dd), 7.55 (1H, td), 7.42 (1H, d), 7.33 (1H, t), 7.11 (1H, d), 7.03 (1H, t), 3.92 (3H, s), 3.87 (m, 4H), 3.66 (m, 4H).

### Example 34

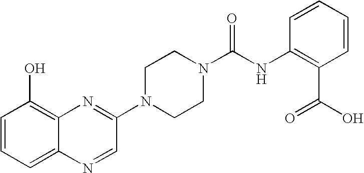

To sodium hydride (48 mg, 1.2 mmol, 60%) in 7 mL of DMF at 0° C. was added dodecanethiol (243 mg, 1.2 mmol). The mixture was warmed to room temperature and stirred for 15 min. To this mixture was added a solution of EXAMPLE 32 (35 mg, 0.086 mmol) in 3 mL of DMF. The mixture was heated at 130° C. for 4 h. The mixture was filtered and purified by Gilson to afford the desired product as a yellow solid. ¹H NMR (acetone-d₆, 500 MHz) δ 11.1 (1H, s), 8.71 (1H, s), 8.65 (1H, d), 8.09 (1H, d), 7.56 (1H, t), 7.49 (1H, t), 7.16 (1H, d), 7.06 (1H, t), 6.84 (1H, d), 3.99 (4H, m), 3.78 (4H, m); LCMS m/z 394 (M+1).

### Synthesis of DP Antagonist Compounds

Numerous DP receptor antagonist compounds have been published and are useful and included in the methods of the present invention. For example, DP receptor antagonists can be obtained in accordance with WO01/79169 published on Oct. 25, 2001, EP 1305286 published on May 2, 2003, WO02/094830 published on Nov. 28, 2002 and WO03/062200 published on Jul. 31, 2003. Compound AB can be synthesized in accordance with the description set forth in WO01/66520A1 published on Sep. 13, 2001; Compound AC can be synthesized in accordance with the description set forth in WO03/022814A1 published on Mar. 20, 2003, and Compounds AD and AE can be synthesized in accordance with the description set forth in WO03/078409 published on Sep. 25, 2003.

The synthesis of the remaining DP antagonist compounds disclosed herein can be undertaken using the description provided in WO2004/103370 published on Dec. 2, 2004.

### Biological Assays

The activity of the compounds of the present invention regarding niacin receptor affinity and function can be evaluated using the following assays:

### ³H-Niacin Binding Assay:

1. Membrane: Membrane preps are stored in liquid nitrogen in:
20 mM HEPES, pH 7.4
0.1 mM EDTA

Thaw receptor membranes quickly and place on ice. Resuspend by pipetting up and down vigorously, pool all tubes, and mix well. Use clean human at 15 μg/well, clean mouse at 10 ug/well, dirty preps at 30 ug/well.

1a. (human): Dilute in Binding Buffer.
1b. (human+4% serum): Add 5.7% of 100% human serum stock (stored at −20° C.) for a final concentration of 4%. Dilute in Binding Buffer.
1c. (mouse): Dilute in Binding Buffer.
2. Wash buffer and dilution buffer: Make 10 liters of ice-cold Binding Buffer:
20 mM HEPES, pH 7.4
1 mM MgCl₂
0.01% CHAPS (w/v)
use molecular grade or ddH₂O water
   3. [5, 6-³H]-nicotinic acid: American Radiolabeled Chemicals, Inc. (cat #ART-689). Stock is ˜50 Ci/mmol, 1 mCi/ml, 1 ml total in ethanol-20 μM
   Make an intermediate ³H-niacin working solution containing 7.5% EtOH and 0.25 μM tracer. 40 μL of this will be diluted into 200 μL total in each well→1.5% EtOH, 50 nM tracer final.
   4. Unlabeled nicotinic acid:
   Make 100 mM, 10 mM, and 80 μM stocks; store at −20° C. Dilute in DMSO.

### 5. Preparing Plates:

1) Aliquot manually into plates. All compounds are tested in duplicate. 10 mM unlabeled nicotinic acid must be included as a sample compound in each experiment.
2) Dilute the 10 mM compounds across the plate in 1:5 dilutions (8 μl:40 μl).
3) Add 195 μL binding buffer to all wells of Intermediate Plates to create working solutions (250 μM→0). There will be one Intermediate Plate for each Drug Plate.
4) Transfer 5 μL from Drug Plate to the Intermediate Plate. Mix 4-5 times.

### 6. Procedure:

1) Add 140 μL of appropriate diluted 19CD membrane to every well. There will be three plates for each drug plate: one human, one human+serum, one mouse.
2) Add 20 μL of compound from the appropriate intermediate plate
3) Add 40 μL of 0.25 μM ³H-nicotinic acid to all wells.
4) Seal plates, cover with aluminum foil, and shake at RT for 3-4 hours, speed 2, titer plate shaker.
5) Filter and wash with 8×200 μL ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid competition binding assay within the range of about 100 nM to about 25 μM.

### ³⁵S-GTPγS Binding Assay:

Membranes prepared from Chinese Hamster Ovary (CHO)-K1 cells stably expressing the niacin receptor or vector control (7 μg/assay) were diluted in assay buffer (100 mM HEPES, 100 mM NaCl and 10 mM MgCl₂, pH 7.4) in Wallac Scintistrip plates and pre-incubated with test compounds diluted in assay buffer containing 40 μM GDP (final [GDP] was 10 μM) for 10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

### Membrane Preparation

### Materials:

| | |
|---|---|
| CHO-K1 cell culture medium: | F-12 Kaighn's Modified |
| | Cell Culture Medium with 10% FBS, |
| | 2 mM L-Glutamine, |
| | 1 mM Sodium Pyruvate |
| | and 400 μg/ml G418 |
| Membrane Scrape Buffer: | 20 mM HEPES |
| | 10 mM EDTA, pH 7.4 |
| Membrane Wash Buffer: | 20 mM HEPES |
| | 0.1 mM EDTA, pH 7.4 |
| Protease Inhibitor Cocktail: | P-8340, (Sigma, St. Louis, MO) |

### Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 uL Protease Inhibitor Cocktail.
Spin at 20,000 rpm for 17 minutes at 4° C.
Aspirate off the supernatant and resuspend pellet in 30 mL Membrane Wash Buffer. Add 50 μL Protease Inhibitor Cocktail.
Spin at 20,000 rpm for 17 minutes at 4° C.
Aspirate the supernatant off the membrane pellet. The pellet may be frozen at −80° C. for later use or it can be used immediately.

### Assay

### Materials:

Guanosine 5′-diphosphate sodium salt (GDP, Sigma-Aldrich Catalog #87127)
Guanosine 5′-[γ³⁵S] thiotriphosphate, triethylammonium salt ([³⁵S]GTPγS, Amersham Biosciences

### Catalog #SJ1320, ˜1000 Ci/mmol)

96 well Scintiplates (Perkin-Elmer #1450-501)

### Binding Buffer: 20 mM HEPES, pH 7.4

100 mM NaCl
10 mM MgCl₂
   GDP Buffer: binding buffer plus GDP, ranging from 0.4 to 40 μM, make fresh before assay

### Procedure:

(total assay volume=100 μ̅well)
25 μL GDP buffer with or without compounds (final GDP 10 μM—so use 40 μM stock)
50 μL membrane in binding buffer (0.4 mg protein/mL)
25 μL [³⁵S]GTPγS in binding buffer. This is made by adding 5 μl [³⁵S]GTPγS stock into 10 mL binding buffer (This buffer has no GDP)
Thaw compound plates to be screened (daughter plates with 5 μL compound @ 2 mM in 100% DMSO)
Dilute the 2 mM compounds 1:50 with 245 μL GDP buffer to 40 μM in 2% DMSO. (Note: the concentration of GDP in the GDP buffer depends on the receptor and should be optimized to obtain maximal signal to noise; 40).
Thaw frozen membrane pellet on ice. (Note: they are really membranes at this point, the cells were broken in the hypotonic buffer without any salt during the membrane prep step, and most cellular proteins were washed away)
Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay.
Dilute membrane to a protein concentrations of 0.40 mg/ml in Binding Buffer. (Note: the final assay concentration is 20 μg/well).
Add 25 μL compounds in GDP buffer per well to Scintiplate.
Add 50 μL of membranes per well to Scintiplate.
Pre-incubate for 5-10 minutes at room temperature. (cover plates with foil since compounds may be light sensitive)
Add 25 μL of diluted [³⁵S]GTPγS. Incubate on shaker (Lab-Line model #1314, shake at setting of 4) for 60 minutes at room temperature. Cover the plates with foil since some compounds might be light sensitive.
Assay is stopped by spinning plates sealed with plate covers at 2500 rpm for 20 minutes at 22° C.
Read on TopCount NXT scintillation counter—35S protocol.

The compounds of the invention generally have an EC₅₀ in the functional in vitro GTPγS binding assay within the range of about less than 1 μM to as high as about 100 μM.

### Flushing via Laser Doppler

Male C57B16 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's baseline and data is normalized to an average of the baseline mean intensities.

### Materials and Methods—Laser Doppler Pirimed PimII; Niacin (Sigma); Nembutal (Abbott Labs).

Compounds of this invention did not display flushing in this assay at doses as high as 100 mg/kg.

Moreover, the nicotinic acid receptor has been identified and characterized in WO02/084298A2 published on Oct. 24, 2002 and in Soga, T. et al., Tunaru, S. et al. and Wise, A. et al. (citations above).

All patents, patent applications and publications that are cited herein are hereby incorporated by reference in their entirety. While certain preferred embodiments have been described herein in detail, numerous alternative embodiments are seen as falling within the scope of the invention.

1. A compound in accordance with formula I: 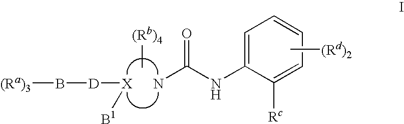
or a pharmaceutically acceptable salt or solvate thereof, is disclosed wherein:
X represents a carbon or nitrogen atom, such that 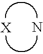
represents a 5 to 7 membered heterocyclic ring containing 1-2 nitrogen atoms;
when X represents a nitrogen atom, D represents a bond and B¹ is absent;
when X represents a carbon atom, B and B¹ can be taken together or separately;
when B and B¹ are taken together, D represents a bond and B and B¹ taken together represent a spiro ring containing 5-6 atoms, optionally containing 1 heteroatom or group selected from oxygen, sulfur, sulfinyl, sulfonyl and nitrogen, said spiro ring being optionally substituted with 1 oxo group, and optionally fused to a phenyl ring, said spiro or fused phenyl ring having 3 R^{a} groups,
and when B and B¹ are taken separately, D represents a bond, an oxygen atom or —(CH₂)₁₋₃—, B¹ represents hydrogen and
B represents a 6-10 membered aryl or a 5-10 membered heteroaryl group containing from 1-4 heteroatoms, 0-4 of which are nitrogen, 0-2 of which are oxygen and 0-1 of which are sulfur;
3 R^{a} groups are present, 1-3 of which are selected from the group consisting of: hydrogen and halo, and 0-2 of which are selected from the group consisting of:
OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;
phenyl, heteroaryl, —O-phenyl and —O-heteroaryl, said phenyl and heteroaryl groups and portions being optionally substituted with 1-3 groups, 1-3 of which are halo atoms and 1-2 of which are selected from the group consisting of: C1-3alkyl, haloC1-3alkyl, OC1-3alkyl, haloC1-3alkoxy, OH, NH₂ and CN;
and C₁₋₃alkyl and OC₁₋₃alkyl, the alkyl portions of which are optionally substituted with 1-3 halo atoms and 1 phenyl or heteroaryl group, said phenyl and heteroaryl being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH, NH₂ and CN;
each R^{b} independently represents hydrogen, halo, C₁₋₃alkyl, haloC₁₋₃alkyl, OC₁₋₃alkyl, haloC₁₋₃alkoxy or OH, or two R^{b} groups may be combined to form a fused 5-6 membered ring, with two such rings being possible; 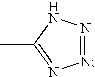
R^{c} represents —CO₂H or
and each R^{d} independently represents H, halo, methyl, or methyl substituted with 1-3 halo atoms.

2. A compound in accordance with claim 1 wherein: D represents a bond, an oxygen atom, —CH₂— or —CH₂CH₂—.

3. A compound in accordance with claim 2 wherein: D represents a bond.

4. (canceled)

5. (canceled)

6. A compound in accordance with claim 1 wherein: 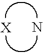
represents a 7 membered heterocyclic ring containing 1-2 nitrogen atoms.

7. (canceled)

8. (canceled)

9. A compound in accordance with claim 1 wherein: each R^{b} is selected from a hydrogen atom and CH₃ or two R^{b} groups are taken in combination and represent a 5 membered ring, with two such rings being present.

10. (canceled)

11. (canceled)

12. (canceled)

13. A compound in accordance with claim 9 wherein two R^{b} groups are taken in combination and represent a 5 membered ring, with two such rings being present.

14. A compound in accordance with claim 1 wherein B and B¹ are taken separately, such that B¹ represents H and B represents a 6-10 membered aryl or a 5-10 membered heteroaryl group containing from 14 heteroatoms, 0-4 of which are nitrogen, 0-2 of which are oxygen and 0-1 of which are sulfur.

15. A compound in accordance with claim 14 wherein B and B¹ are taken separately, B¹ represents H and B represents a 6-10 membered aryl group.

16. A compound in accordance with claim 15 wherein B represents a naphthyl group.

17. A compound in accordance with claim 14 wherein B and B¹ are taken separately, B¹ represents H and B represents a 5-10 membered heteroaryl group.

18. A compound in accordance with claim 1 wherein B and B¹ are taken together and represent a spiro ring having 5-6 atoms.

19. A compound in accordance with claim 18 wherein B and B¹ are taken together and represent a spiro ring having 5 or 6 atoms one of which is an oxygen atom.

20. (canceled)

21. (canceled)

22. (canceled)

23. A compound in accordance with claim 1 represented by formula I-A: 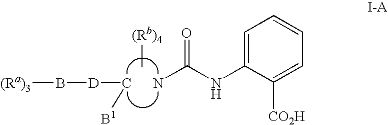
or a pharmaceutically acceptable salt or solvate thereof, wherein:
D represents a bond, an oxygen atom, —CH2- or —CH2CH2-;
each R^{b} is selected from a hydrogen atom and CH3 or two R^{b} groups are taken in combination and represent a 5 membered ring, with two such rings being present;
B and B¹ can be taken together or separately;
when B and B¹ are taken together, B and B¹ taken together represent a spiro ring containing 5-6 atoms, optionally containing 1 heteroatom or group selected from oxygen, sulfur, sulfinyl, sulfonyl and nitrogen, said spiro ring being optionally substituted with 1 oxo group, and optionally fused to a phenyl ring, said spiro or fused phenyl ring having 3 R^{a} groups,
and when B and B¹ are taken separately, B¹ represents hydrogen and
B represents a 6-10 membered aryl or a 5-10 membered heteroaryl group containing from 1-4 heteroatoms, 0-4 of which are nitrogen, 0-2 of which are oxygen and 0-1 of which are sulfur;
and 0-1 R^{a} groups are selected from
OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;
phenyl, heteroaryl, —O-phenyl and —O-heteroaryl, said phenyl and heteroaryl groups and portions being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC₁₋₃alkoxy, OH, NH₂ and CN;
and C1-3alkyl and OC₁₋₃alkyl, the alkyl portions of which are optionally substituted with 1-3 halo atoms and 1 phenyl or heteroaryl group, said phenyl and heteroaryl being optionally substituted with 1-3 groups, 1-3 of which are halo atoms and 1-2 of which are selected from the group consisting of: C₁₋₃alkyl, haloC₁₋₃alkyl, OC₁₋₃alkyl, haloC1-3alkoxy, OH, NH2 and CN,
and the remaining 2-3 R^{a} groups are selected from H and halo.

24. A compound in accordance with claim 1 represented by formula I-B: 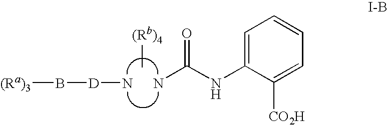
or a pharmaceutically acceptable salt or solvate thereof, wherein:
D represents a bond;
each Rb is selected from a hydrogen atom and CH3 or two Rb groups are taken in combination and represent a 5 membered ring, with two such rings being present;
B represents a 6-10 membered aryl or a 5-10 membered heteroaryl group containing from 1-4 heteroatoms, 0-4 of which are nitrogen, 0-2 of which are oxygen and 0-1 of which are sulfur;
and 0-1 Ra groups are selected from
OH; NH₂; NHC₁₋₃ alkyl; N(C₁₋₃alkyll)₂; CN; C(O)NH₂; C(O)NH(C₁₋₃alkyl; C(O)N(C₁₋₃alkyl)₂;
and the remaining 2-3 R^{a} groups are selected from H and halo.

25. A compound in accordance with claim 24 wherein B represents a 10 membered aryl or a 9-10 membered heteroaryl group containing from 1-4 heteroatoms, 0-4 of which are nitrogen, 0-2 of which are oxygen and 0-1 of which is sulfur, said group B being substituted with 3 R^{a} groups, one of which is OH and the remainder of which are hydrogen or halo atoms.

26. A compound in accordance with claim 1 as set forth below in Table 1:
TABLE 1 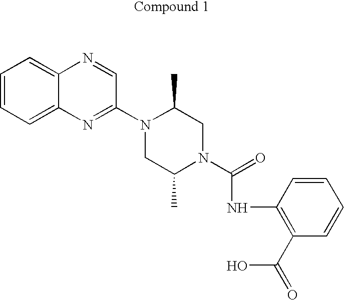 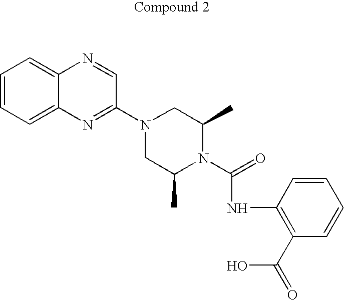 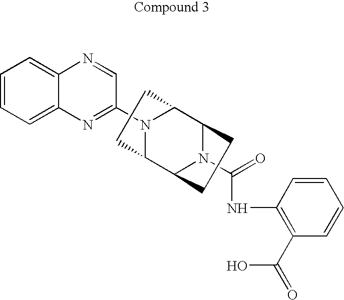 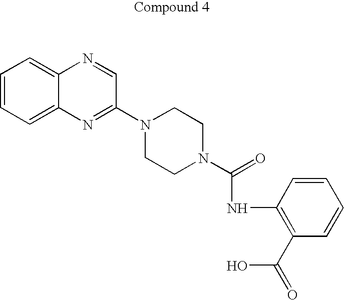 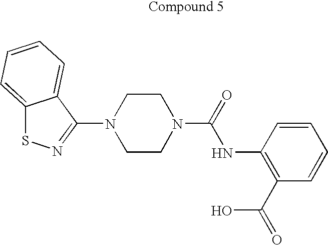 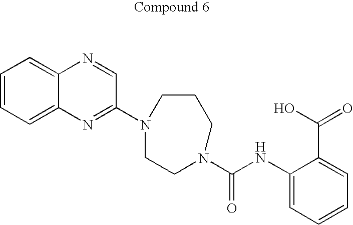 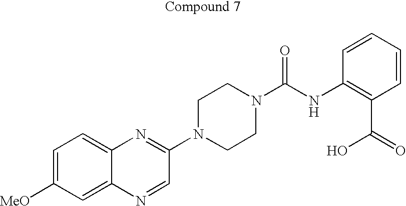 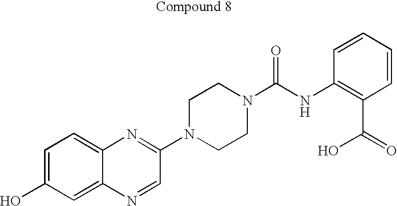 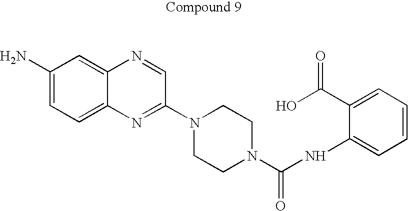 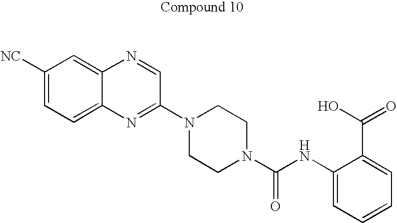 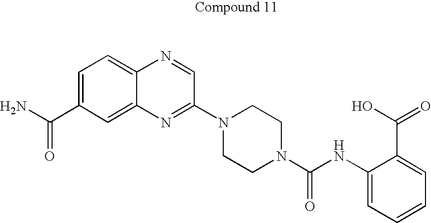 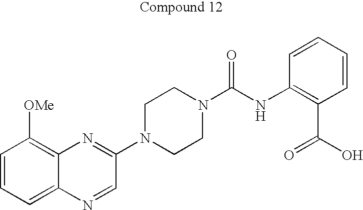 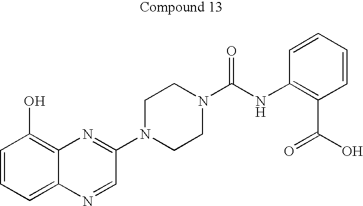 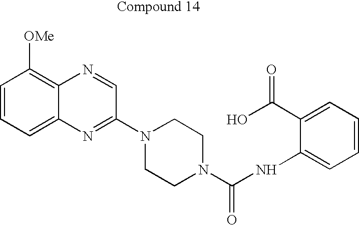 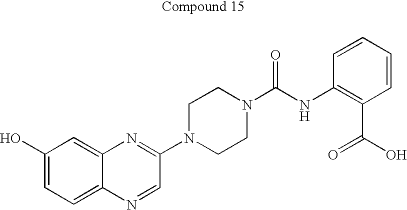 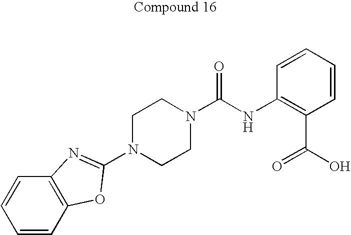 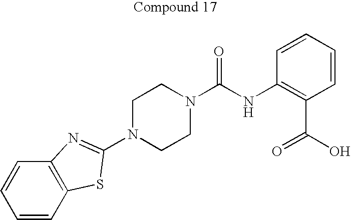 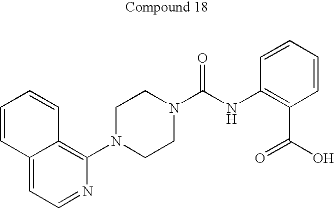 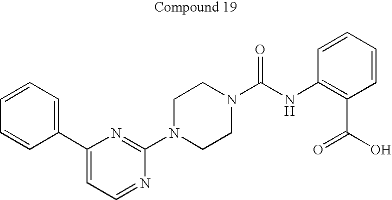 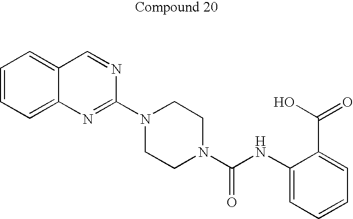 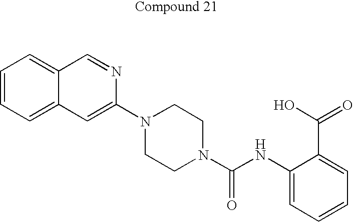 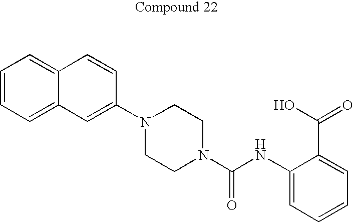 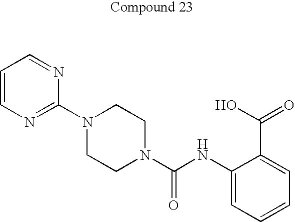 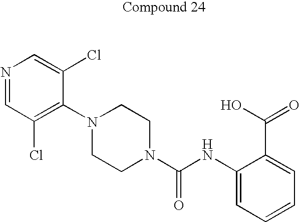 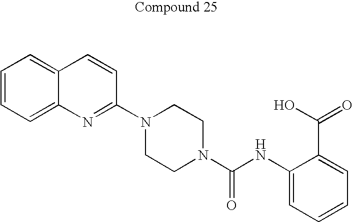 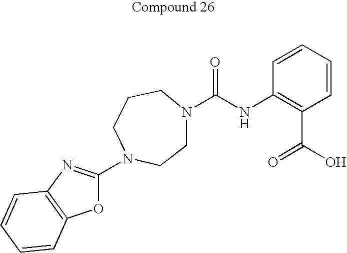 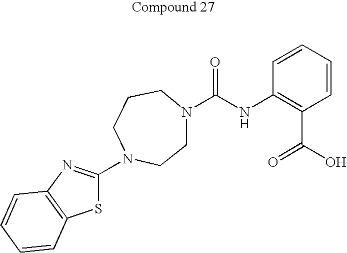 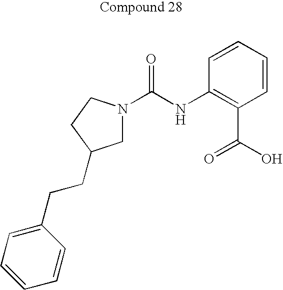 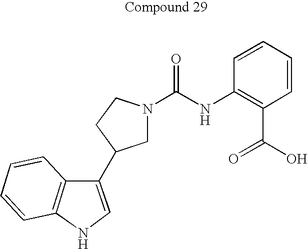 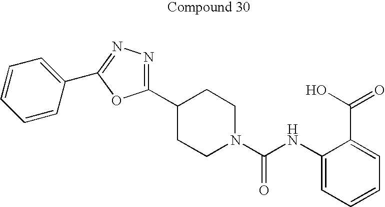 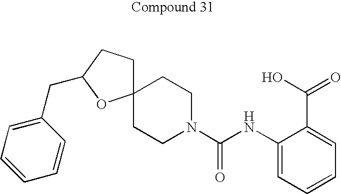 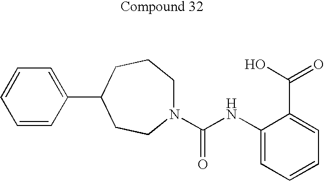 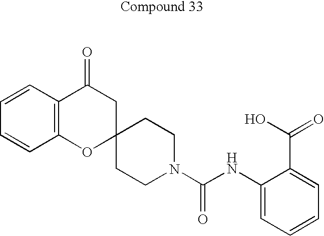 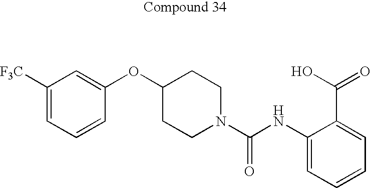
or a pharmaceutically acceptable salt or solvate thereof.

27. A pharmaceutical composition comprised of a compound in accordance with claim 1 in combination with a pharmaceutically acceptable carrier.

28. A method of treating atherosclerosis in a human patient in need of such treatment comprising administering to the patient a compound of claim 1 in an amount that is effective for treating atherosclerosis.

29. A method of treating dyslipidemia in a human patient in need of such treatment comprising administering to the patient a compound of claim 1 in an amount that is effective for treating dyslipidemias.

30. A method of treating diabetes in a human patient in need of such treatment comprising administering to the patient a compound of claim 1 in an amount that is effective for treating diabetes.

31. A method of treating metabolic syndrome in a human patient in need of such treatment comprising administering to the patient a compound of claim 1 in an amount that is effective for treating metabolic syndrome.

32. A method of treating atherosclerosis, dyslipidemias, diabetes, metabolic syndrome or a related condition in a human patient in need of such treatment, comprising administering to the patient a compound of claim 1 and a DP receptor antagonist, said combination being administered in an amount that is effective to treat atherosclerosis, dyslipidemia, diabetes or a related condition in the absence of substantial flushing.

33. A method of treating atherosclerosis, dyslipidemias, diabetes or a related condition in a human patient in need of such treatment, comprising administering to the patient a compound of claim 1 and a DP receptor antagonist selected from the group consisting of compounds A through AJ: 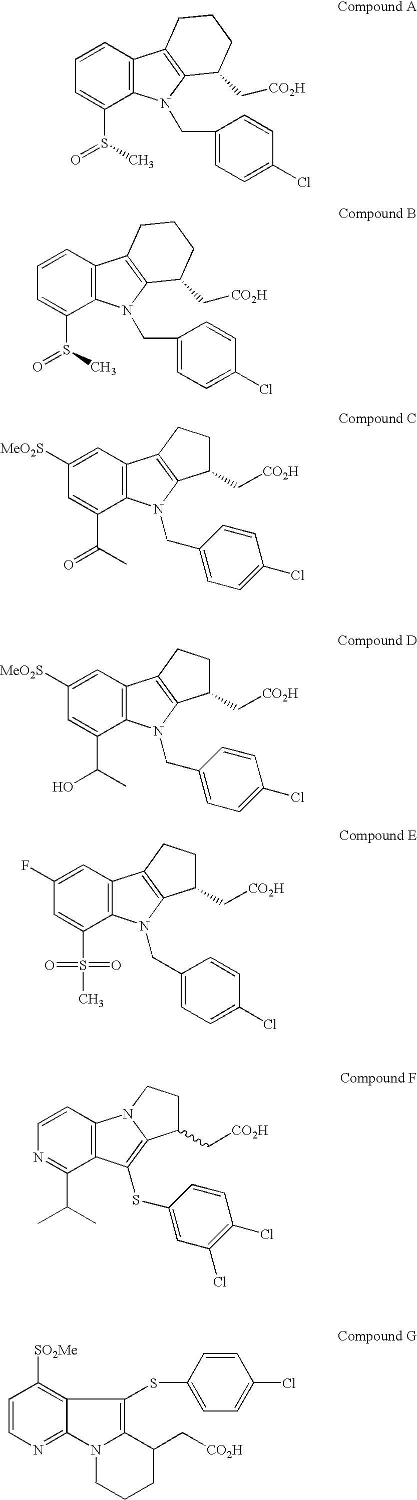 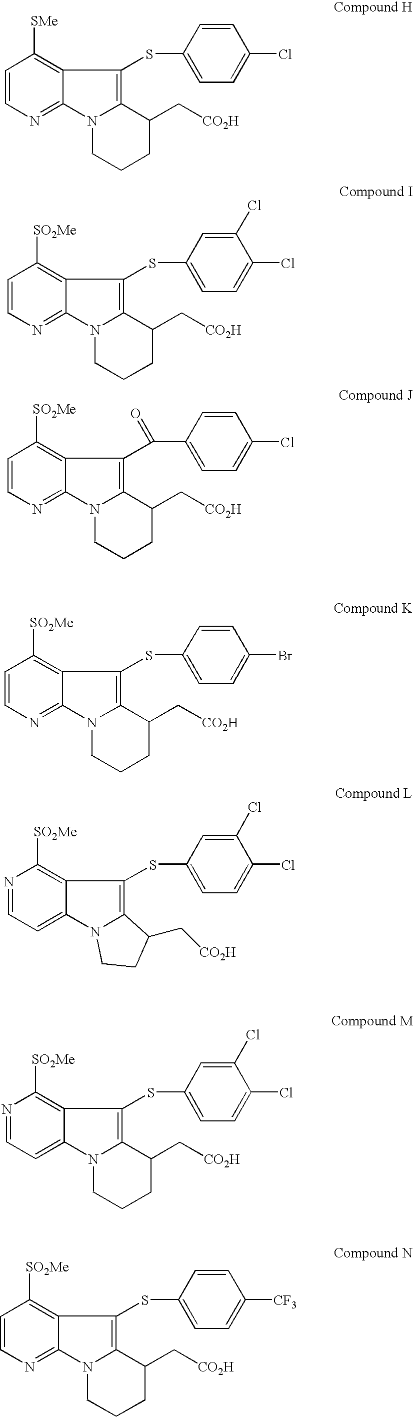 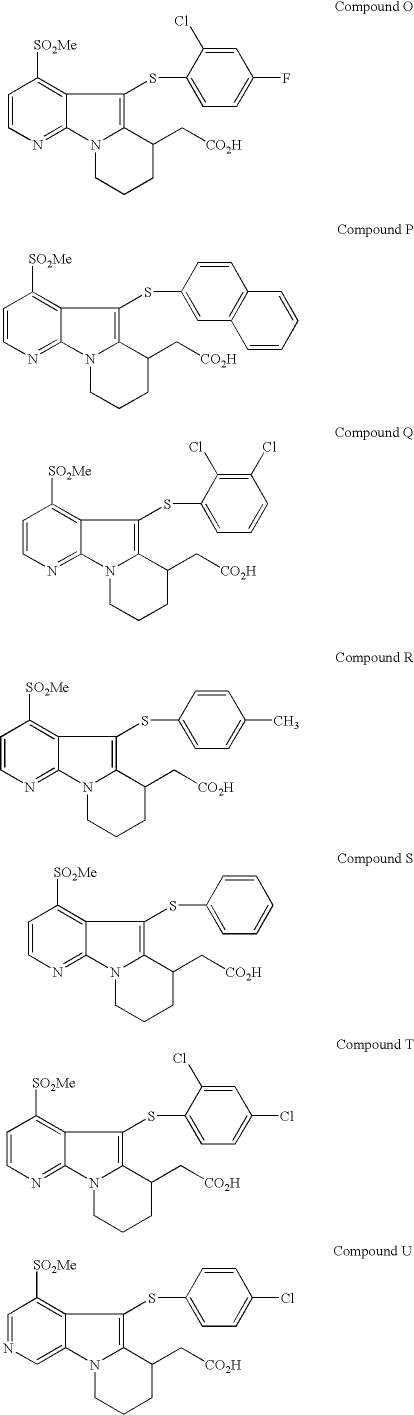 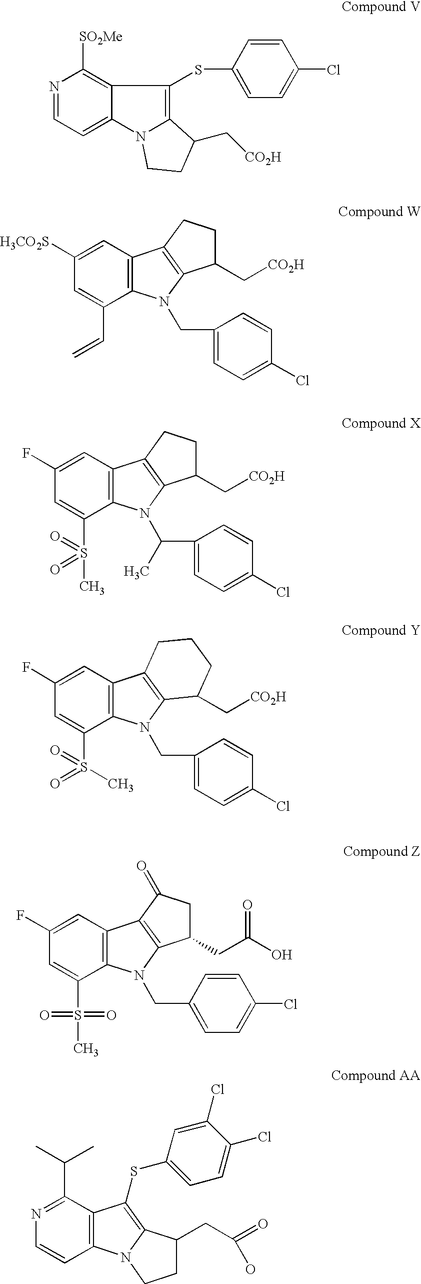 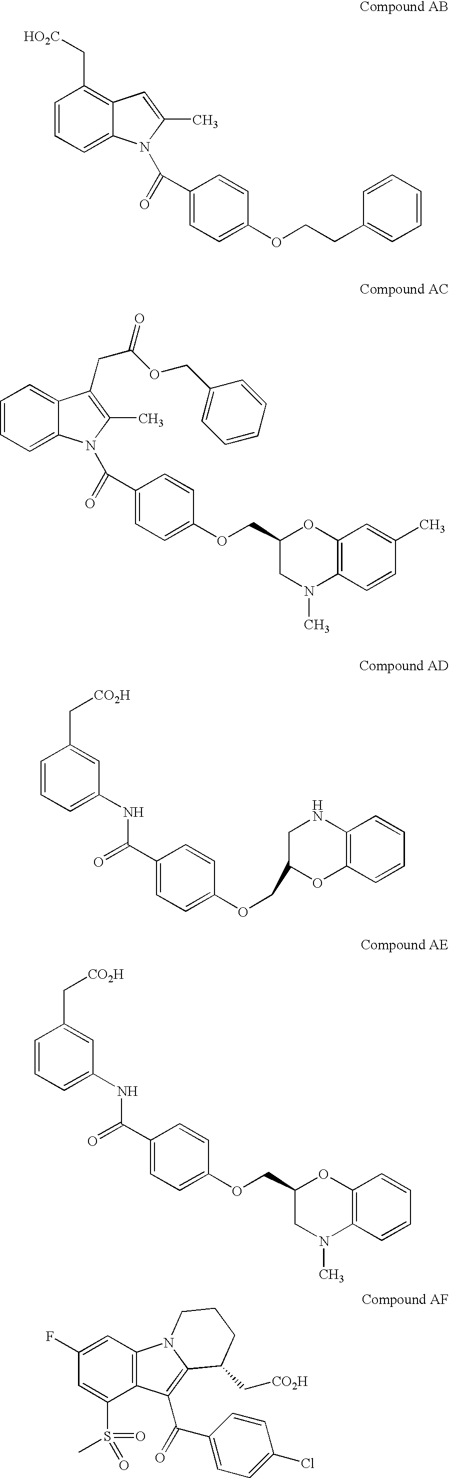 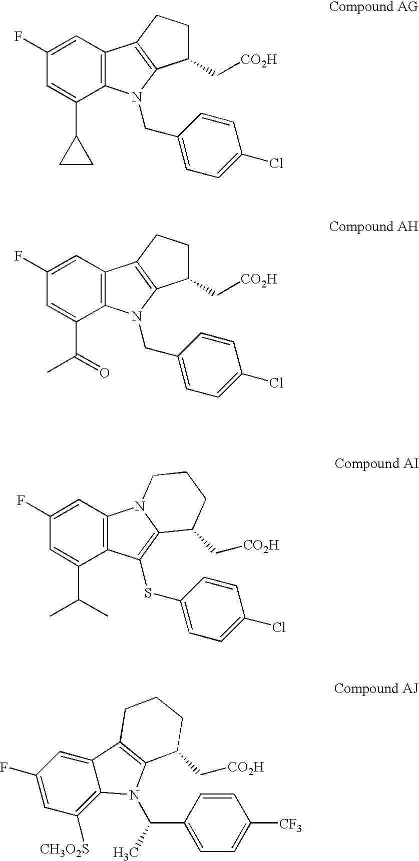
or a pharmaceutically acceptable salt or solvate thereof.
